SEQUENCE LISTING (18 sequences)

SEQ ID NO: 1 - DNA, 22 bp, Mus musculus
cccagtctctgatattctgtac

SEQ ID NO: 2 - DNA, 22 bp, Mus musculus
tccgcgccgtttgctggagaaa

SEQ ID NO: 3 - DNA, 22 bp, Photinus pyralis
ctgtgccagagtccttcgatag

SEQ ID NO: 4 - DNA, 18 bp, Homo sapiens
cgcagagtgtaccggaga

SEQ ID NO: 5 - DNA, 18 bp, Homo sapiens
tgcccaatgtgaagcagt

SEQ ID NO: 6 - DNA, 18 bp, Photinus pyralis
tgcgctgctggtgccaac

SEQ ID NO: 7 - DNA, 23 bp, Artificial Sequence
  PCR Forward Primer B2m-F1
accggcctgtatgctatccagaa

SEQ ID NO: 8 - DNA, 23 bp, Artificial Sequence
  PCR Reverse Primer B2m-R2
ccatactggcatgcttaactctg

SEQ ID NO: 9 - DNA, 23 bp, Artificial Sequence
  PCR Forward Primer Bcat1-F1
tgtggctgtacggcaaggacaac

SEQ ID NO: 10 - DNA, 22 bp, Artificial Sequence
  PCR Reverse Primer Bcat1-R2
gtagctcgattgtccagtcact

SEQ ID NO: 11 - DNA, 23 bp, Artificial Sequence
  PCR Forward Primer mB2m-F
accggcctgtatgctatccagaa

SEQ ID NO: 12 - DNA, 22 bp, Artificial Sequence
  PCR Reverse Primer mB2m-R
aatgtgaggcgggtggaactgt

SEQ ID NO: 13 - DNA, 21 bp, Artificial Sequence
  PCR Forward Primer hB2M-F
atgagtatgcctgccgtgtga

SEQ ID NO: 14 - DNA, 20 bp, Artificial Sequence
  PCR Reverse Primer hB2M-R
ggcatcttcaaacctccatg

SEQ ID NO: 15 - DNA, 20 bp, Artificial Sequence
  PCR Forward Primer hBCAT1-F
tggagaatggtcctaagctg

SEQ ID NO: 16 - DNA, 20 bp, Artificial Sequence
  PCR Reverse Primer hBCAT1-R
gcacaattgtccagtcgctc

SEQ ID NO: 17 - DNA, 20 bp, Artificial Sequence
  PCR Forward Primer hMYC-F
gagcaaggacgcgactctcc

SEQ ID NO: 18 - DNA, 20 bp, Artificial Sequence
  PCR Reverse Primer hMYC-R
gcaccgagtcgtagtcgagg

(19) United States
(12) Patent Application Publication
(10) Pub. No.: US 2019/0240176 A1
(43) Pub. Date: Aug. 8, 2019

(54) METHODS OF TREATMENT FOR MYELOID LEUKEMIA

(71) Applicant: The University of Georgia Research Foundation, Inc., Athens, GA (US)

(72) Inventors: Takahiro Ito, Athens, GA (US); Ayuna Hattori, Athens, GA (US)

(21) Appl. No.: 16/344,492

(22) PCT Filed: Oct. 26, 2017
(86) PCT No.: PCT/US2017/058457

§ 371 Date: Apr. 24, 2019

Related U.S. Application Data
(60) Provisional application No. 62/413,028, filed on Oct. 26, 2016.

(51) Int. Cl. A61K 31/197; A61K 31/436; C07C 229/28; A61P 35/02

(52) U.S. Cl. CPC A61K 31/197; A61K 31/436; A61K 45/06; A61P 35/02; C07C 229/28

(57) ABSTRACT

Embodiments of the present disclosure provide for methods of treating cancer (e.g., leukemia), pharmaceutical compositions for treating cancer, methods of modulating cancer progression and development, and the like.

### CROSS-REFERENCE TO RELATED APPLICATIONS

This application claims the benefit of and priority to U.S. Provisional Application Ser. No. 62/413,028, having the title “METHODS OF TREATMENT FOR MYELOID LEUKEMIA”, filed on Oct. 26, 2016, the disclosure of which is incorporated herein by reference in its entirety.

### SEQUENCE LISTING

The instant application contains a sequence listing which has been submitted with the instant application via EFS-Web. The sequence listing file is named 222102-2780_ST25.txt and is incorporated herein by reference in its entirety.

### BACKGROUND

Reprogrammed cellular metabolism is a common characteristic observed in various cancers. Yet it remains poorly understood whether metabolic changes directly regulate cancer development and progression. Cells sense intrinsic and extrinsic nutrient status and respond by modulating metabolic processes to control their proliferation and differentiation. It is evident that deregulated cell energetics is one of the hallmarks of cancer and that cancer cells alter their metabolic processes to meet the biosynthetic demands of rapid growth and to enhance their fitness in the tumor microenvironment. The discovery of aerobic glycolysis, known as the Warburg effect, provided the initial clue to help the understanding of how the metabolism of tumor cells differs from non-tumor cells. Cancer metabolism research in the past decade has revealed that aerobic glycolysis is just one example of the metabolic alterations in cancer and that such alterations can serve as therapeutic targets. Importantly, many of the metabolic changes in cancer are not passive, but rather active; in fact, oncogenic events can directly reprogram cellular processes in glucose, fatty acid and amino acid metabolisms. Ras mutations are associated with enhanced glucose usage via activation of the glucose transporter GLUT1. Another example is the oncogenic transcription factor c-Myc and its role in glutamine (Gln) metabolism. Although Gln is not an essential amino acid, the growth of Myc-expressing tumor cells is dependent upon this amino acid. Gln not only affects proliferation but also modulates cell differentiation in cancer. It has long been known that glutaminase is an effective treatment for several malignancies by lowering the plasma Gln level, and increased serum amino acid levels are often correlated with poor prognosis in multiple cancers. Recent studies have identified indispensable roles of the serine biosynthetic pathway in lung and breast cancers. Despite the accumulating evidence, it remains uncertain whether these metabolic alterations directly regulate cancer development and progression in vivo.

### SUMMARY

Embodiments of the present disclosure provide for compositions and methods for treating chronic myeloid leukemia, compositions and methods for modulating cancer progression, and the like.

An embodiment of the present disclosure includes a method of treating blast crisis condition in chronic myeloid leukemia in a subject, including administering to the subject a therapeutically effective amount of a composition including a tyrosine kinase inhibitor, gabapentin (or derivatives thereof), and rapamycin (or derivatives thereof), or a pharmaceutically acceptable salt or a prodrug thereof.

An embodiment of the present disclosure includes a method of modulating cancer progression and development, including administering to the subject a therapeutically effective amount of a composition including a tyrosine kinase inhibitor, gabapentin (or derivatives thereof), and rapamycin (or derivatives thereof), or a pharmaceutically acceptable salt or a prodrug thereof, wherein the composition interrupts the branched-chain amino acid transamination pathway.

An embodiment of the present disclosure includes a pharmaceutical composition including a therapeutically effective amount of a composition which contains a tyrosine kinase inhibitor, gabapentin (or derivatives thereof), and rapamycin (or derivatives thereof), or a pharmaceutically acceptable salt of the composition or a prodrug of the composition, and a pharmaceutically acceptable carrier.

An embodiment of the present disclosure includes a method of treating blast crisis condition in chronic myeloid leukemia in a subject, including: administering to the subject a therapeutically effective amount of each of a tyrosine kinase inhibitor, gabapentin (or derivatives thereof), and rapamycin (or derivatives thereof), or a pharmaceutically acceptable salt or a prodrug thereof.

An embodiment of the present disclosure includes a method of modulating cancer progression and development, comprising administering to the subject a therapeutically effective amount of each of a tyrosine kinase

Other compositions, methods, features, and advantages will be or become apparent to one with skill in the art upon examination of the following drawings and detailed description. It is intended that all such additional compositions, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-J demonstrate activated BCAA production by BCAT1 in BC-CML. FIG. 1A shows intracellular amino acid levels in CP-CML (n=7, open bars) and BC-CML (n=9, closed bars). Amounts per 2×10⁵ cells. FIG. 1B shows Bcat1 expression in normal and leukemic hematopoietic cells. Serial cDNA dilutions were used for RT-PCR analysis. Normal LSK cells, CP- and BC-CML cells, M1 myeloid cells and no reverse transcriptase (−RT) and water controls are shown. B2m, beta-2-microglobulin. FIG. 1C shows BCAT1 protein expression in primary mouse leukemia (n=3 each). FIG. 1D is a schematic of an embodiment of the reaction catalyzed by BCAT1. KG, alpha-ketoglutarate. FIGS. 1E-H graph intracellular Val production from KIV captured by ¹H-¹³C HSQC analysis. FIGS. 1E and 1F show one dimensional (top) and two dimensional (bottom) HSQC spectra. The amounts of (FIG. 1G) ¹³C-KIV and (FIG. 1H) ¹³C-Val produced per one million cells are shown (n=3 each). FIG. 1I shows ¹⁵N-amine incorporation into BCAAs. Amounts per one million cells. n=3 per time point. FIG. 1J shows BCAT1-dependent production of BCAAs. The amounts of ¹⁵N-BCAAs produced are normalized with that of ¹⁵N-Glu/Gln. n=3 each. *p<0.05, **p<0.01, ***p<0.001 by two-tailed t-test. All data are mean±s.e.m.

FIGS. 2A-G demonstrate that Bcat1 is essential for BC-CML propagation and differentiation arrest. FIGS. 2A-B demonstrate the colony-forming ability of primary Lin⁻ BC-CML cells (FIG. 2A) transduced with the indicated shRNAs, or (FIG. 2B) plated with the indicated concentrations of gabapentin or vehicle (−). 1,000 cells/well (n=3). Photomicrographs show representative colonies formed under each condition. Scale bar, 500 μm. FIG. 2C illustrates Bcat1 knockdown impaired BC-CML development in vivo. BC-CML cells expressing the indicated constructs were transplanted, and the survival of the recipients was monitored. shCtrl, n=20; shBcat1-a, n=19; shBcat1-b, n=16. FIGS. 2D and 2G show the percentage of immature myeloblasts in leukemic mice. Photomicrographs of Wright's stained leukemia cells. Arrowheads, immature myeloblasts; arrows, differentiating myelocytes and mature band cells. Scale bar, 10 μm. FIG. 2e shows a survival curve of mice serially transplanted with Lin⁻ cells from primary shRNA-expressing leukemias. 1,000 cells/mouse (1k), n=10 for shCtrl, n=8 for shBcat1-a; and 3,000 cells/mouse (3k), n=9 for shCtrl, n=10 for shBcat1-a. FIG. 2F shows a survival curve of mice transplanted with LSK cells infected with BCR-ABL1 and the vector-control or Bcat1. n=17 each. Inset, spleens from the indicated groups. Error bars indicate s.e.m. *p<0.05, **p<0.01, ***p<0.001 by two-tailed t-test (FIGS. 2D and 2G) or log-rank test (FIGS. 2C, 2E, and 2F).

FIGS. 3A-K illustrate BCAT1 activation and requirement in human myeloid leukemia. FIG. 3A shows BCAT1 expression in healthy subjects (n=5) and in patients with chronic and blast crisis CML (n=4 each) at IMSUT Hospital, the University of Tokyo. FIGS. 3B-C provide microarray data analysis of (FIG. 3B) BCAT1 and (FIG. 3C) BCAT2 expression in 57 chronic (gray), 15 accelerated (pink) and 41 blast crisis (blue) phase patients. The bars represent the normalized expression in each specimen. FIGS. 3D-E demonstrate colony-forming ability of primary human BC-CML cells treated with (FIG. 3D) shBCAT1 or (FIG. 3) Gbp. n=3 each. Two independent patient specimens were tested. FIG. 3F shows BCAT1 expression in healthy subjects and de novo AML patients at IMSUT Hospital (n=5). FIG. 3G demonstrates colony-forming ability of Gbp-treated primary human AML cells. FIG. 3H is a Kaplan-Meier analysis of overall survival in the AML patient cohorts with low (bottom quartile) or high (top quartile) BCAT1 expression. n=93 in each cohort. FIG. 3I shows that BCAA supplementation augmented the colony-forming ability of BCAT1-knockdown K562 cells (n=3). FIG. 3J shows Western blotting for the indicated proteins. K562 cells treated with shBCAT1-d or 20 mM Gbp for 24 h. (−), shCtrl or vehicle controls. FIG. 3K shows the effect of BCAA on mTORC1 pathway activation in BCAT1-knockdown K562 cells. Cells were treated with or without BCAA or rapamycin, and analyzed at 24 h post-treatment. Error bars indicate s.e.m. NS, not statistically significant (p>0.05). *p<0.05, **p<0.01, ***p<0.001 by two-tailed t-test (FIGS. 3D, 3E, 3G, 3I) or log-rank test (FIG. 3H).

FIGS. 4A-F demonstrate RNA binding protein MSI2 mediates BCAT1 activation in BC-CML. FIGS. 4A-B show RNA immunoprecipitation (RIP) with (FIG. 4A) anti-FLAG antibody from K562 cells expressing empty vector, FLAG-tagged MSI2 (WT) or FLAG-MSI2 with defective RNA binding domains (RBD), or (FIG. 4B) RIP with anti-MSI2 (αMSI2) or a control IgG (nIgG) from K562 cells. Co-immunoprecipitated RNAs were analyzed for the enrichment of BCAT1, beta-2-microglobulin (B2M) and c-MYC transcripts. n=3 each. FIGS. 4C-D show the effect of (FIG. 4C) BCAT1 overexpression and (FIG. 4D) BCAA supplementation on the colony-forming ability of MSI2-knockdown K562 cells (n=3). (−), shCtrl or vehicle controls. FIG. 4E shows the effect of nutrient supplementation on mTORC1 pathway activation in MSI2-knockdown K562 cells. FIG. 4F is an embodiment of the MSI2-BCAT1-BCAA axis in BC-CML. Error bars indicate s.e.m. *p<0.05, **p<0.01, by two-tailed t-test.

FIGS. 5A-L illustrate change in the amino acid metabolism in leukemic mice. FIGS. 5A-D are representative chromatograms of CP-CML (FIGS. 5A, 5C) and BC-CML (FIGS. 5B, 5D) plasma samples derivatized with the amine-specific fluorescent labeling agent NBD-F and analyzed in mobile phases at pH 6.2 (FIGS. 5A, 5B) or pH 4.4 (FIGS. 5C, 5D). Each NBD-amino acid peak is assigned as indicated. IS, internal standard (NBD-6-aminocaproic acid). FIG. 5E shows plasma amino acid levels in mice with CP- and BC-CML. Blood plasma samples were prepared from mice with CP- and BC-CML, methanol-extracted and dried under a vacuum. The dried extracts were analyzed for quantification. Open and closed bars indicate CP-CML (n=5) and BC-CML (n=7) specimens, respectively. Two-tailed t-test. †p<0.06, *p<0.05, **p<0.01. FIG. 5F shows leucine transport in primary CP- and BC-CML cells. BCR-ABL1-YFP⁺PI⁻ live leukemia cells (5×10⁵) were sorted from premorbid animals and placed in a pre-warmed uptake media containing 10 μM [(U)-¹⁴C]-labeled L-leucine. After incubation at 37° C. for the indicated times, the cells were washed with cold HBSS and lysed with 0.1 M sodium hydroxide, and the radioactivity was measured using a scintillation counter. The gray and blue lines indicate the average leucine uptake in CP- and BC-CML samples, (n=5 and 3, respectively). Error bars indicate s.e.m. *p<0.05. NS, not statistically significant (p>0.05). FIG. 5G provides RT-qPCR analysis of Bcat1 and Bcat2 expression in CP- and BC-CML cells (n=4 each). The expression levels are normalized and displayed relative to the control beta-2-microglobulin gene expression. Error bars indicate s.e.m.; ***p<0.001, NS, not statistically significant (p>0.05). FIG. 5H shows BCAT1 protein expression in mouse primary CP- and BC-CML cells. This graph shows BCAT1 protein expression levels normalized relative to the HSP90 loading control (CP-CML, n=7; BC-CML, n=9). Error bars indicate s.e.m. *p<0.05. FIG. 5I demonstrates tissue-specific expression of mouse Bcat1. The expression was detectable in the myeloid cell line M1, primary mouse BC-CML cells, olfactory bulb (Olf bulb), whole brain and testis. B2m, beta-2-microglobulin. FIG. 5J is one embodiment of the structures of human and mouse BCAT1 proteins. The shaded boxes represent aminotransferase domains. K, a Lys residue for the binding of the pyridoxal phosphate cofactor. CVVC, a conserved redox-sensitive CXXC motif. Regions targeted with shRNAs in this study are shown as thick bars (shBcat1-a and -b, and shBCAT1-c and -d). FIGS. 5K-L show alanine and aspartate transaminase gene expression in CP- and BC-CML. RT-qPCR analysis of (FIG. 5K) Gpt1 and Gpt2, and (FIG. 5L) Got1 and Got2 expression in CP- and BC-CML samples (n=4 each). The expression levels are normalized and displayed relative to the expression of the B2m control. Error bars indicate s.e.m.; NS, not statistically significant (p>0.05).

FIGS. 6A-E show keto acid metabolism in leukemic mice. FIGS. 6A-B are representative chromatograms of CP- (FIG. 6A) and BC-CML (FIG. 6B) plasma samples derivatized with the keto acid-reactive o-phenylenediamine (OPD). Each OPD-keto acid peak is assigned as indicated. KG, alpha-ketoglutarate; PYR, pyruvate; KIV, keto-isovalerate; KIC, keto-isocaproate; KMV, keto-methylvalerate. IS, internal standard for keto acid analysis (OPD-alpha-ketovalerate). FIGS. 6C-D show plasma and intracellular branched-chain keto acid levels in CP- and BC-CML. Blood plasma fractions from leukemic mice (FIG. 6C) or FACS-purified live leukemia cells (5×10⁶) (FIG. 6D) were methanol-extracted and dried under a vacuum. The dried extracts were labeled with OPD, extracted with ethyl acetate and analyzed using an HPLC system equipped with a fluorescence detector. Open and closed bars indicate CP-CML (plasma, n=9; intracellular, n=5) and BC-CML (plasma, n=10; intracellular, n=6) specimens, respectively. BCKAs, total branched-chain keto acids. *p<0.05. Error bars indicate s.e.m. FIG. 6E shows the molar amount of intracellular BCAAs and BCKAs in primary mouse BC-CML cells. The amount of each organic acid per one million cells is estimated using calibration curves obtained with reference standards for each compound. “% KA/AA” shows the amount of a BCKA relative to the corresponding BCAA species.

FIGS. 7A-I show intracellular BCAA production from BCKA in human K562 BC-CML cells. FIGS. 7A-F shows regions of HSQC spectra of ¹³C-labeled metabolites. K562 cells were cultured in media supplemented with 170 μM ¹³C-Val and 30 μM non-labeled KIV (FIGS. 7A, 7C), or 170 μM non-labeled Val and 30 μM ¹³C-KIV (FIGS. 7B, 7D). After labeling for 15 min, the cells were collected, washed with PBS and methanol-extracted for HSQC analysis by high-field NMR spectroscopy. Each panel shows the regions of 1-dimensional and 2-dimensional HSQC spectra for the intracellular fraction (FIGS. 7A, 7B), culture supernatant (FIGS. 7C, 7D), and labeling media alone (FIGS. 7E, 7F), respectively. FIGS. 7A and 7B are the same as shown in FIGS. 1E and 1F, respectively. FIGS. 7G-I bsence of detectable intracellular KIC generation by Leu breakdown. K562 cells were cultured in the labeling medium supplemented with 170 μM ¹³C-Leu and 30 μM non-labeled KIC for 15 min, and the intracellular ¹³C-labeled metabolites were analyzed by HSQC analysis. Each panel shows region of the 2D spectrum showing ¹H-¹³C HSQC signals for beta, gamma and delta carbons of Leu and KIC. (FIG. 7G) intracellular fraction, (FIG. 7H) KIC reference standard (HSQC signals derived from natural abundance ¹³C-KIC), (FIG. 7I) overlay of the spectra FIG. 7G (black) and FIG. 7H (red). Note the absence of KIC signals in (FIG. 7G).

FIGS. 8A-G shows intracellular BCAA production via transamination. FIGS. 8A-C are regions of 600 MHz 2D HMBC spectra showing crosspeaks between the amine nitrogen and the beta carbon protons. Only those amino acids that have incorporated a significant amount of ¹⁵N-amine show crosspeak signals. FIGS. 8D-F are regions of 600 MHz 1D ¹H spectra. Each proton peak is assigned as indicated. DSS, 2,2-dimethyl-2-silapentane-5-sulfonate. FIGS. 8A and 8D are a mixture of reference standards of the indicated amino acids, (FIGS. 8B, 8E) K562 cell sample cultured in the medium containing (amine-¹⁵N)-glutamine and (FIGS. 8C, 8F) K562 cell sample cultured in the non-labeled standard medium. FIG. 8G shows the percentage of newly synthesized ¹⁵N-labeled BCAAs within total intracellular pool at 72 h after post-labeling for each amino acid indicated. “Total BCAAs” shows the percentage including all three BCAA species.

FIGS. 9A-M show the roles of Bcat1 in differentiation, proliferation and leukemia development in vivo. FIG. 9A provides RT-qPCR analysis of Bcat1 expression. Lin⁻ cells from NUP98-HOXA9/BCR-ABL-induced BC-CML were infected with shCtrl or Bcat1 shRNA (shBcat1-a and shBcat1-b) for 3 days and resorted for analysis of Bcat1 expression. The expression levels are normalized to the level of B2m expression and displayed relative to the control, which was arbitrarily set at 1. Error bars represent s.e.m. of triplicate PCRs. **p<0.01. FIG. 9B provides RT-qPCR analysis of Bcat1 expression in leukemia cells isolated from diseased mice transplanted with shCtrl- or shBcat1-expressing BC-CML cells. The expression levels are normalized and displayed relative to the B2m control. ***p<0.001. FIG. 9C shows Bcat2 expression in shBcat1-expressing cells. Lin⁻ cells from NUP98-HOXA9/BCR-ABL-induced BC-CML were infected with shCtrl or Bcat1 shRNA (shBcat1-a and shBcat1-b) for 3 days and resorted for analysis of Bcat2 expression. The expression levels are normalized to the level of B2m and are displayed relative to the control arbitrarily set at 1. Error bars represent the s.e.m. of triplicate PCRs. NS, not statistically significant (p>0.05). FIG. 9D shows the functional rescue of the shBcat1-induced reduction in colony-forming ability with the expression of shRNA-resistant mutant Bcat1 cDNA. Primary Lin⁻ BC-CML cells transduced with the vector or shRNA-resistant Bcat1 gene together with the indicated shRNA constructs. **p<0.01 compared with the vector and shBcat1-b. e, f, FIGS. 9E and 9F show colony-forming ability of primary HSPCs. FIG. 9E shows normal HSPCs purified from bone marrow based on their LSK phenotype were transduced with the Bcat1 shRNAs (shBcat1-a and shBcat1-b) and plated for colony formation. NS, not statistically significant (p>0.05). FIG. 9F shows normal HSPCs were plated for colony formation with the indicated concentrations of gabapentin or PBS (−). NS, not statistically significant (p>0.05). **p<0.01 compared with the PBS control. Photomicrographs showing representative colonies formed under each condition. Scale bar, 500 μm. 300 LSK cells were plated per well in triplicate for the evaluation of colony-forming activity. Error bars indicate s.e.m. FIG. 9G shows hematoxylin and eosin staining of sections of the liver, lung and spleen at the time of onset of clinical signs (top 6 rows) and of tissue sections from a disease-free survivor (bottom 2 rows). White arrows indicate immature myeloid cells. Portal triad (PT), hemorrhagic necrosis (N), central veins (CV), arteriolar profiles (A), bile ducts (B), veins (V), white pulp (WP) and red pulp (RP) are indicated. Scale bars, 100 μm for images at 10× and 20 μm for images at 40× magnification. FIG. 9H provides representative flow cytometry plots showing lineage marker expression in leukemia cells from mice transplanted with the shRNA-infected BC-CML cells. Leukemia cells were analyzed for their frequency of the Lin⁻ population. FIGS. 9I-K show the effect of conditional Bcat1 knockdown on BC-CML maintenance in vivo. In FIG. 9I Lin⁻ BC-CML cells were infected with doxycycline-inducible shRNAs against shBcat1-b or a control (shCtrl) and transplanted into recipients (1,500 cells per recipient). After ten days of the transplantation with leukemia cells expressing the indicated constructs, In FIG. 9J, donor-derived chimerisms were analyzed. Mice were then fed with chow containing doxycycline to induce shRNA expression, and survival was monitored (FIG. 9K). The data shown are from two independent experiments. n=4 for shCtrl with no Dox (DOX−); n=7 for shBcat1-b, DOX−; and n=9 each for shCtrl with Dox (DOX+) and shBcat1-b, DOX+. **p<0.01 (shCtrl vs shBcat1-b, DOX+). NS, not statistically significant (p>0.05). FIG. 9L shows cell cycle distribution of the shRNA-infected leukemia cells. Live leukemia cells were isolated from mice transplanted with shRNA-infected BC-CML cells, fixed and stained with propidium iodide for analysis of cell cycle distribution via flow cytometry. FIG. 9M shows apoptotic cells from leukemic mice transplanted with shRNA-infected BC-CML cells were analyzed via flow cytometry using Annexin V and 7-aminoactinomycin D (7-AAD) staining.

FIGS. 10A-G demonstrate that BCAT1 cooperates with BCR-ABL1 in blastic transformation in vivo. FIG. 10A shows RT-qPCR analysis of Bcat1 expression in normal LSK or Lin⁻ c-Kit⁺ HSPCs transduced with either the vector or Bcat1 retroviruses. The expression levels are normalized and displayed relative to the control B2m expression. ***p<0.001. FIG. 10B shows normal LSK or Lin⁻ c-Kit⁺ HSPCs that were purified from healthy bone marrow and transduced with the indicated retroviruses, and the infected cells were plated in triplicate to assess colony formation after 10 days. Error bars indicate s.e.m. NS, not statistically significant (p>0.05). FIG. 10C shows colony-forming ability of normal HSPCs expressing BCR-ABL1 and Bcat1. LSK cells were purified from healthy bone marrow and transduced with either the control vector or Bcat1 together with BCR-ABL1 (B/A) retroviruses, and double-positive cells were plated in triplicate to assess colony formation after 10 days (plated at a density of 150 cells/well). Photomicrographs showing representative colonies formed in each group. Scale bar, 500 μm. Error bars indicate s.e.m. ***p<0.001. FIG. 10D shows chimerism of donor-derived cells after transplantation with LSK cells expressing the indicated constructs. n=15 for each group. *p<0.05. FIG. 10E shows hematoxylin and eosin staining of liver, lung and spleen sections from mice transplanted with LSK cells expressing BCR-ABL1 and vector or Bcat1. White arrows indicate immature myeloid cells. Scale bars, 100 μm for 10× images and 20 μm for 40× images. FIG. 10F shows plasma α-amino acid levels in mice transplanted with LSK cells infected with BCR-ABL1 and the vector or Bcat1. Blood plasma fractions were prepared from peripheral blood samples, methanol-extracted and dried under a vacuum. The dried extracts were labeled with NBD-F and analyzed using an HPLC equipped with a fluorescence detector. Open and closed bars indicate vector-control (n=17) and Bcat1 (n=18) specimens, respectively. *p<0.05, **p<0.01. FIG. 10G provides representative flow cytometry plots showing lineage marker expression in leukemia cells from mice transplanted with LSK cells infected with either the control vector or Bcat1 together with BCR-ABL1. Leukemia cells were analyzed for their frequency of the Lin⁻ population.

FIGS. 11A-J demonstrate that BCAT1 is required for human myeloid leukemia. FIG. 11A shows RT-qPCR analysis of BCAT1 expression in the human K562 BC-CML cell line transduced with lentiviral shCtrl or BCAT1 shRNA (shBCAT1-c and shBCAT1-d). The expression levels are normalized and displayed relative to the expression of the B2M control. **p<0.01. FIG. 11B shows Western blot analysis of BCAT1 protein levels in K562 cells infected with the indicated lentiviral shRNA constructs. Human β-tubulin protein (β-Tub) was used as the loading control. β-Tub image is the same as shown in FIG. 3j. FIGS. 11c, 11D show colony-forming ability of (FIG. 11C) K562 cells transduced with control (shCtrl) or BCAT1 shRNAs (shBCAT1-c and shBCAT1-d) and (FIG. 11D) K562 cells cultured with the indicated concentrations of Gbp. One hundred cells were plated per well in triplicate. Photomicrographs show representative colonies formed. Scale bar, 200 μm. Error bars indicate s.e.m. **p<0.01, ***p<0.001. FIG. 11E shows RT-qPCR analysis of BCAT1 expression in the samples from the BC-CML patient used in the data presented in FIG. 3d that were transduced with control (shCtrl) or BCAT1 shRNA (shBCAT1-d). ***p<0.001. FIG. 11F shows colony-forming ability of primary human CD34⁺ BC-CML cells from another patient specimen treated with Gbp. Error bars indicate s.e.m. **p<0.01. FIG. 11G-I show colony-forming ability of MV4-11 (FIG. 11G), U937 (FIG. 11H) and HL60 human AML cells (FIG. 11I) treated with the indicated concentrations of Gbp. MV4-11, HL60 cells (300/well) or U937 (100/well) were plated in triplicate. Photomicrographs show representative colonies formed. Scale bars, 200 μm. Error bars indicate s.e.m. *p<0.05, **p<0.01, ***p<0.001. FIG. 11J shows BCAT1 expression in human de novo AML patients. Data for BCAT1 expression levels from the TCGA AML dataset were divided into quartiles and were compared. On average, top quartile cohort showed 1.6-fold higher expression level than the bottom quartile. **p<0.01.

FIGS. 12A-D show the impact of BCAT1 knockdown in K562 cells.

FIGS. 12A,-B show the effect of BCAT1 knockdown (FIG. 12A) or Gbp treatment (FIG. 12B) on the intracellular concentrations of glutamate and BCAAs in K562 cells. The shCtrl or PBS control values were set as 100%. Error bars indicate s.e.m. n=10 each for (FIG. 12A) and n=3 each for (FIG. 12B). **p<0.01. FIG. 12C shows the AKT activation status in BCAT1- or MSI2-knockdown K562 cells. K562 cells treated with shCtrl, shBCAT1 or shMSI2 were analyzed by Western blotting for phospho-AKT (at Thr308 or Ser473), total AKT, hBCAT1, hMSI2 and HSP90 levels. FIG. 12D shows the effect of alpha-ketoglutarate supplementation on the colony-forming ability of BCAT1-knockdown cells. K562 cells transduced with shCtrl (−) or shBCAT1-d (+) were plated in triplicate with or without 1 mM dimethyl-alpha-ketoglutarate (KG) and/or 4 mM BCAAs as indicated. n=3 technical replicates. Error bars indicate s.e.m. *p<0.05, **p<0.01. NS, not statistically significant (p>0.05).

FIGS. 13A-D show MSI2 and BCAT1 expression in human cancer. FIG. 13A is a microarray data analysis of MSI2 expression in human CML. Gene expression data of chronic (gray, n=57), accelerated (pink, n=15) and blast crisis (blue, n=41) phase patients were retrieved from the NCBI GEO database (GSE4170). The bar represents the normalized expression value in each specimen. FIG. 13B provides co-expression analysis of the BCAT1 and MSI2 genes in human cancer. Pearson correlation coefficients were used to evaluate the extent of co-expression patterns. FIG. 13C is an embodiment of the human BCAT1 transcript. The bars represent the putative MSI binding elements (MBEs; r(G/A)U₁₋₃AGU). Forty MBEs were identified within the 3′-UTR of BCAT1. CDS, coding sequence for hBCAT1 protein. FIG. 13D shows K562 cells infected with lentiviral shRNA against MSI2 (shMSI2) or shCtrl (−) were analyzed by Western blotting for phospho-S6 kinase (at Thr389; pS6K), total S6K, hMSI2 and HSP90 levels. Note that MSI2 knockdown reduced the levels of BCAT1 protein and phospho-S6K.

FIGS. 14A-B show inhibitory effects of leukemic colony formation by Gabapentin. FIG. 14A shows that BCAT1 inhibition by Gabapentin attenuates leukemic colony formation by human blast crisis CML in combination with the tyrosine kinase inhibitor Imatinib. FIG. 14B shows inhibition of leukemic colony formation by gabapentin (Gbp) and its structural analogs.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, or the like, as such can vary. It is also to be understood that the terminology used herein is for purposes of describing particular embodiments only, and is not intended to be limiting. It is also possible in the present disclosure that steps can be executed in different sequence where this is logically possible.

As used in the specification and claims, the singular forms “a,” “an” and “the” include plural references unless the context clearly dictates otherwise. For example, the term “a cell” includes a plurality of cells, including mixtures thereof.

### Definitions

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g., gorilla or chimpanzee), and rodents such as rats and mice.

In the context of certain aspects of the disclosure, the term “subject” generally refers to an individual who will receive or who has received treatment (e.g., administration of a composition of the disclosure, and optionally one or more other agents) for a condition characterized by a cancer (e.g., leukemia). In certain aspects, a subject may be a healthy subject. Typical subjects for treatment include persons afflicted with or suspected of having or being pre-disposed to a disease disclosed herein, or persons susceptible to, suffering from or that have suffered a disease disclosed herein. A subject may or may not have a genetic predisposition for a disease disclosed herein.

When referring to a subject or patient, the term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-peritoneal, intra-synovial, intrasternal, intrathecal, intrahepatic, intralesional, and intracranial injections or infusion techniques. In some embodiments, the administration is intracaviteal.

The term “diagnosed” as used herein, refers to the recognition of a disease by its signs and symptoms (e.g., resistance to conventional therapies), or genetic analysis, pathological analysis, histological analysis, and the like.

The terms “administering” and “administration” as used herein refer to a process by which a therapeutically effective amount of a composition of the disclosure are delivered to a subject for prevention and/or treatment purposes. Compositions are administered in accordance with good medical practices taking into account the subject's clinical condition, the site and method of administration, dosage, patient age, sex, body weight, and other factors known to physicians.

The terms “administration of” and “administering” a compound or composition as used herein refers to providing a compound of the disclosure or a prodrug of a compound of the disclosure to the individual in need of treatment. The compounds of the present disclosure may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The terms “treat” or “treatment” as used herein refer to both therapeutic treatment and prophylactic or preventative measures, where the object is to prevent or slow down (lessen) an undesired physiological change or disorder resulting from the disease. Beneficial or desired clinical results include, but are not limited to, alleviation of symptoms, diminishment of the extent of disease, stabilized (i.e., not worsening) state of disease, and delay or slowing of progression of the symptoms recognized as originating from a stroke. The term “treatment” can also refer to prolonging survival as compared to expected survival if not receiving treatment. Those in need of treatment include those already with the condition or disorder as well as those prone to have the condition or disorder or those in which the condition or disorder is to be prevented or onset delayed.

As used herein, the terms “prophylactically treat” and “prophylactically treating” refer completely or partially preventing a disease or symptom thereof and/or may be therapeutic in terms of a partial or complete cure for a disease and/or adverse effect attributable to the disease.

The term “modulate” refers to the activity of a composition to affect (e.g., to promote or retard) an aspect of cellular function, including, but not limited to, cell growth, proliferation, apoptosis, and the like.

The term “unit dosage form,” as used herein, refers to physically discrete units suitable as unitary dosages for human and/or animal subjects, each unit containing a predetermined quantity of a compound or composition calculated in an amount sufficient to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound or composition in the subject.

As used herein, a “pharmaceutical composition” and a “pharmaceutical formulation” are meant to encompass a composition suitable for administration to a subject, such as a mammal, especially a human. In general, a “pharmaceutical composition” or “pharmaceutical formulation” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

A “pharmaceutically acceptable excipient”, “pharmaceutically acceptable diluent”, “pharmaceutically acceptable carrier”, and “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one or more such excipients, diluents, carriers, and adjuvants.

The terms “therapeutically effective amount” and “an effective amount” are used interchangeably herein and refer to that amount of the composition being administered that is sufficient to effect the intended application including but not limited to disease treatment. For example, an effective amount of the composition will relieve to some extent one or more of the symptoms of the disease being treated, and/or that amount that will prevent, to some extent, one or more of the symptoms of the disease that the host being treated has or is at risk of developing. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on the particular composition chosen, the dosing regimen to be followed, whether it is administered in combination with other compounds, timing of administration, the tissue to which it is administered, and the physical delivery system in which it is carried.

“Pharmaceutically acceptable salt” refers to those salts (organic or inorganic) that retain the biological effectiveness and optionally other properties of the free bases. Pharmaceutically acceptable salts can be obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

In the event that embodiments of the disclosed compositions form salts, these salts are within the scope of the present disclosure. Reference to a composition of any of the formulas herein is understood to include reference to salts thereof, unless otherwise indicated. The term “salt(s)”, as employed herein, denotes acidic and/or basic salts formed with inorganic and/or organic acids and bases. In addition, when composition contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of the composition may be formed, for example, by reacting the composition or compounds of the composition with an amount of acid or base, such as an equivalent amount, in a medium such as one in which the salt precipitates or in an aqueous medium followed by lyophilization.

Embodiments of the composition that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, malates (salts formed with malic acid), maleates (formed with maleic acid), ethanesulfonates (formed with ethanesulfonic acid), methanesulfonates (formed with methanesulfonic acid), 2-naphthalenesulfonates, nicotinates, nitrates, oxalates, pectinates, persulfates, 3-phenylpropionates, phosphates (formed with phosphoric acid), picrates, pivalates, propionates, salicylates, succinates, sulfates (such as those formed with sulfuric acid), sulfonates (such as those mentioned herein including those formed with p-toluenesulfonic acid), tartrates, thiocyanates, toluenesulfonates such as tosylates, undecanoates, and the like.

Embodiments of the composition that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with compounds such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Solvates of the compounds of the disclosure are also contemplated herein.

The term “prodrug” refers to an inactive precursor of a composition that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet, 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab, 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

### Abbreviations

Gbp, gabapentin; Pgb, pregabalin; Gaba, gamma-aminobutyric acid; TI-1, 2-(1-Aminocyclohexyl)acetic acid hydrochloride; TI-3, 4-Amino-3-phenylbutanoic acid; JK-1, Tranexamic acid.

### Discussion:

Embodiments of the present disclosure provide for methods of treating cancer (e.g. leukemia), pharmaceutical compositions for treating cancer, methods of modulating cancer progression and development, and the like. Embodiments of the present disclosure can be used to treat blast crisis phase chronic myelogenous leukemia (BC-CML), which is fundamentally different from chronic phase chronic myelogenous leukemia (CP-CML) and is characterized by differentiation arrest and propagation of immature progenitor cells, resistance to current treatments because of secondary mutations with a poor prognosis and shorter median survival. Embodiments of the present disclosure include a combination or “cocktail” approach to treating BC-CML using a mix of different compounds.

In an embodiment, a method of treating blast crisis condition in chronic myeloid leukemia in a subject includes administering to a subject of need of treatment a therapeutically effective amount of a composition. In an embodiment, the composition includes a tyrosine inhibitor, gabapentin or derivative thereof, and rapamycin or derivative thereof, or a pharmaceutically acceptable salt or a prodrug of the composition and/or one or more components of the composition. In an embodiment, the amount of each of the tyrosine inhibitor, gabapentin, and rapamycin (inclusive of derivatives of one or more of these) can be about 1 to 50 weight percent or about 1 to 35 weight percent.

In one embodiment, the composition components are administered as individual components by the same route of administration or by different routes of administration, with administration of each component or components at substantially the same time or at times frames that achieve the desired outcome. In one embodiment, the composition components are formulated into a “cocktail composition”, using methods known by one skilled in the art.

A “tyrosine kinase inhibitor” is meant a molecule that inhibits the function or the production of one or more tyrosine kinases. Tyrosine kinase inhibitors include small molecule inhibitors of tyrosine kinases, antibodies to tyrosine kinases, and antisense oligomers and RNAi inhibitors that reduce the expression of tyrosine kinases.

In an embodiment, the tyrosine kinase inhibitors can include tyrosine kinase inhibitors used to treat CP-CML. In an embodiment, the tyrosine kinase inhibitors can include one or more of the following: curcumin, difluorinated curcumin (DFC), [3-{5-[4-cyclopentyloxy)-2-hydroxybenzoyl]-2-[(3-hydroxy-1,2-benzisoxazol-6-yl) methoxy]phenyl}propionic acid] (T5224, Roche), nordihydroguaiaretic acid (NDGA), dihydroguaiaretic acid (DHGA), [(E,E,Z,E)-3-methyl-7-(4-methylphenyl)-9-(2,6,6-trimethyl-1-cyclohexen-1-yl)-2,4,6,8-nonatetraenoic acid (SR 1302, Tocris Biosciences), (E)-2-benzylidene-3-(cyclohexylamino)-2,3-dihydro-1H-inden-1-one (BCI), TPI-2, TPI-3, triptolide, genistein, imatinib mesylate (Gleevec®), nilotinib (Tasigna®), dasatinib (Sprycel®), ponatinib (Iclusig®), leflunomide (SU101), ZD1839 (Iressa®), OSI-774 (Tarceva®), CI-1033, SU5416, SU6668, ZD4190, ZD6474, PTK787, PKI166, GW2016, EKB-509, EKB-569, CEP-701, CEP-751, PKC412, SU11248, MLN518, trastuzumab (Herceptin®), C225 (Fibitux®), rhu-Mab VEGF (Avastin®), MDX-H210, 2C4, MDX-447, IMC-ICI 1, EMD 72000, RH3, and ABX-EGF. For each of the tyrosine kinase inhibitors, pharmaceutically acceptable salts or a prodrugs can be used as well.

In an embodiment, gabapentin or derivatives of gabapentin can be used in the composition, where the derivatives of gabapentin, when combined with the two other components, can obtain similar or the same results. For each of the gabapentin or derivatives of gabapentin, pharmaceutically acceptable salts or prodrugs can be used as well.

In an embodiment, gabapentin refers to 1-(aminomethyl)cyclohexane acetic acid and derivatives of gabapentin as well as pharmaceutically acceptable salts, esters, solvates, hydrates, and polymorphs thereof, can also be used in the composition. (See also U.S. Pat. Nos. 4,024,175, 4,087,544, 4,894,476, 4,960,931 and 6,683,112 for various gabapentin derivatives, which are included herein by reference) 1-(aminomethyl)cyclohexane acetic acid is a γ-aminobutyric acid (GABA) analogue with a molecular formula of C₉H₁₇NO₂ and a molecular weight of 171.24. 1-(aminomethyl)cyclohexane acetic acid is freely soluble in water and in both basic and acidic aqueous solutions. I-(aminomethyl)cyclohexane acetic acid has a structure of:

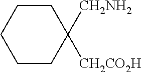

Gabapentin may be obtained from a variety of commercial sources, such as Shanghai Zhongxi International Trading Co., Shanghai, China; Hikal Limited, Bangalore, Karnaraka, India; Erregierre S.p.A., San Paolo d'Argon (BG), Italy; MediChem, SA, Sant Joan Despi (Barcelona), Spain; Ranbaxy Laboratories, New Delhi, India; Procos S.p.A., Gamed, Italy; Zambon Group, Milan, Italy; Hangzhuo Chiral Medicine Chemicals Co., Hangzhuo, China; InterChem Corporation USA, Paramus, N.J.; SST Corporation, Clifton, N.J.; Teva Pharmaceuticals USA, North Whales, Pa.; Plantex USA, Hakensack, N.J.; and Sigma-Aldrich, St. Louis, Mo., or an appropriate distributor.

In an embodiment, rapamycin or derivatives of rapamycin can be used in the composition, where the derivatives of rapamycin, when combined with the two other components, can obtain similar or the same results. For each of the rapamycin or derivatives of rapamycin, pharmaceutically acceptable salts or a prodrugs can be used as well.

Rapamycin, in addition to naturally occurring forms of rapamycin, includes rapamycin analogs and derivatives. Many such analogs and derivatives are known in the art. Examples include those compounds described in U.S. Pat. Nos. 6,329,386; 6,200,985; 6,117,863; 6,015,815; 6,015,809; 6,004,973; 5,985,890 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489.595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; all of which are incorporated herein by reference. In particular, rapamycin can include, CCI-779, Everolimus (also known as RADOOI), and ABT-578. CCI-779 is an ester of rapamycin (42-ester with 3-hydroxy-2-hydroxymethyl-2-methylpropionic acid), disclosed in U.S. Pat. No. 5,362,718. Everolimus is an alkylated rapamycin (40-O-(2-hydroxyethyl)-rapamycin, disclosed in U.S. Pat. No. 5,665,772.

In an embodiment, a method of modulating cancer progression and development a subject includes administering to a subject of need of treatment a therapeutically effective amount of the composition. In an embodiment, the composition includes a tyrosine inhibitor, gabapentin or derivative thereof, and rapamycin or derivative thereof, or a pharmaceutically acceptable salt or a prodrug of the composition and/or one or more components of the composition. In an embodiment, the cancer can include BC-CML. Additional details regarding the specific way in which the composition can modulate cancer progression is described in detail in Example 1.

In an embodiment, a pharmaceutical composition comprising a therapeutically effective amount of a composition and a pharmaceutically acceptable carrier. In an embodiment, the composition includes a tyrosine inhibitor, gabapentin or derivative thereof, and rapamycin or derivative thereof, or a pharmaceutically acceptable salt or a prodrug of the composition and/or one or more components of the composition. In an embodiment, the pharmaceutical composition can be used to treat a disease such as cancer (e.g., BC-CML and Acute Myeloid Leukemia).

In various embodiments, the ratios of tyrosine inhibitor, gabapentin or derivative thereof, and rapamycin or derivative thereof can be about 50:200:5 to about 50:67:5, about 50:200:5, or about 50:67:5. In a particular aspect, an illustrative ratios are as follows: Imatinib 50 mg/kg, Gabapentin 200 mg/kg and rapamycin 5 mg/kg) and Imatinib 50 mg/kg, Gabapentin 67 mg/kg and rapamycin 5 mg/kg

### Pharmaceutical Formulations and Routes of Administration

Embodiments of the present disclosure include a compound (e.g., tyrosine inhibitor, gabapentin or derivative thereof, and rapamycin or derivative thereof) as identified herein and formulated with one or more pharmaceutically acceptable excipients, diluents, carriers and/or adjuvants. In addition, embodiments of the present disclosure include a compound formulated with one or more pharmaceutically acceptable auxiliary substances. In particular the compound can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the compound can be administered to the subject using any means capable of resulting in the desired effect. Thus, the compound can be incorporated into a variety of formulations for therapeutic administration. For example, the compound can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the compound may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the compound can be utilized in aerosol formulation to be administered via inhalation. Embodiments of the compound can be formulated into pressurized acceptable propellants such as dichlorodifluoromethane, propane, nitrogen and the like.

Furthermore, embodiments of the compound can be made into suppositories by mixing with a variety of bases such as emulsifying bases or water-soluble bases. Embodiments of the compound can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the compound in a composition as a solution in sterile water, normal saline or another pharmaceutically acceptable carrier.

Embodiments of the compound can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles in accordance with the present disclosure.

In an embodiment, the compound can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the compound can be accomplished using any of a variety of refillable, pump systems. Pumps provide consistent, controlled release over time. In some embodiments, the compound can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electro-osmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, the composition can be delivered using an implantable drug delivery system, e.g., a system that is programmable to provide for administration of the composition. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the compound are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the compound may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the compound described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the composition (e.g., tyrosine inhibitor, gabapentin or derivative thereof, and rapamycin or derivative thereof) can be administered to a subject in one or more doses. Those of skill will readily appreciate that dose levels can vary as a function of the specific the composition administered, the severity of the symptoms and the susceptibility of the subject to side effects. Preferred dosages for a given composition are readily determinable by those of skill in the art by a variety of means and are well above those amounts that might be found in some food products.

In an embodiment, multiple doses of the composition are administered. The frequency of administration of the composition can vary depending on any of a variety of factors, e.g., severity of the symptoms, and the like. For example, in an embodiment, the composition can be administered once per month, twice per month, three times per month, every other week (qow), once per week (qw), twice per week (biw), three times per week (tiw), four times per week, five times per week, six times per week, every other day (god), daily (qd), twice a day (qid), or three times a day (tid). As discussed above, in an embodiment, the composition is administered continuously.

The duration of administration of the composition analogue, e.g., the period of time over which the composition is administered, can vary, depending on any of a variety of factors, e.g., patient response, etc. For example, the composition in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

### Routes of Administration

Embodiments of the present disclosure provide methods and compositions (e.g., tyrosine inhibitor, gabapentin or derivative thereof, and rapamycin or derivative thereof) for the administration of the composition to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration can be combined, if desired, or adjusted depending upon the composition and/or the desired effect. The composition can be administered in a single dose or in multiple doses.

Embodiments of the composition can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the composition. Where systemic delivery is desired, administration typically involves invasive or systemically absorbed topical or mucosal administration of pharmaceutical preparations.

In an embodiment, the composition can also be delivered to the subject by enteral administration. Enteral routes of administration include, but are not limited to, oral and rectal (e.g., using a suppository) delivery.

Methods of administration of the composition through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Examples

### Introduction

Reprogrammed cellular metabolism is a common characteristic observed in various cancers^{1,2}. However, whether metabolic changes directly regulate cancer development and progression remains poorly understood. Embodiments of the present disclosure show that BCAT1, a cytosolic aminotransferase for the branched-chain amino acids (BCAAs), is aberrantly activated and functionally required for chronic myeloid leukemia (CML). BCAT1 is up-regulated during CML progression and promotes BCAA production in leukemia cells by aminating the branched-chain keto acids. Blocking BCAT1 expression or enzymatic activity induces cellular differentiation and impairs the propagation of blast crisis CML (BC-CML) both in vitro and in vivo. Stable isotope tracer experiments combined with NMR-based metabolic analysis demonstrate the intracellular production of BCAAs by BCAT1. Direct supplementation with BCAAs ameliorates the defects caused by BCAT1 knockdown, indicating that BCAT1 exerts its oncogenic function via BCAA production in BC-CML cells. Importantly, BCAT1 expression not only is activated in human BC-CML and de novo acute myeloid leukemia but also predicts disease outcome in patients. As an upstream regulator of BCAT1 expression, Musashi2 (MSI2) was identified as an oncogenic RNA binding protein that is required for BC-CML. MSI2 is physically associated with the BCAT1 transcript and positively regulates its protein expression in leukemia. Taken together, the present disclosure reveals that altered BCAA metabolism activated through the MSI2-BCAT1 axis drives cancer progression in myeloid leukemia.

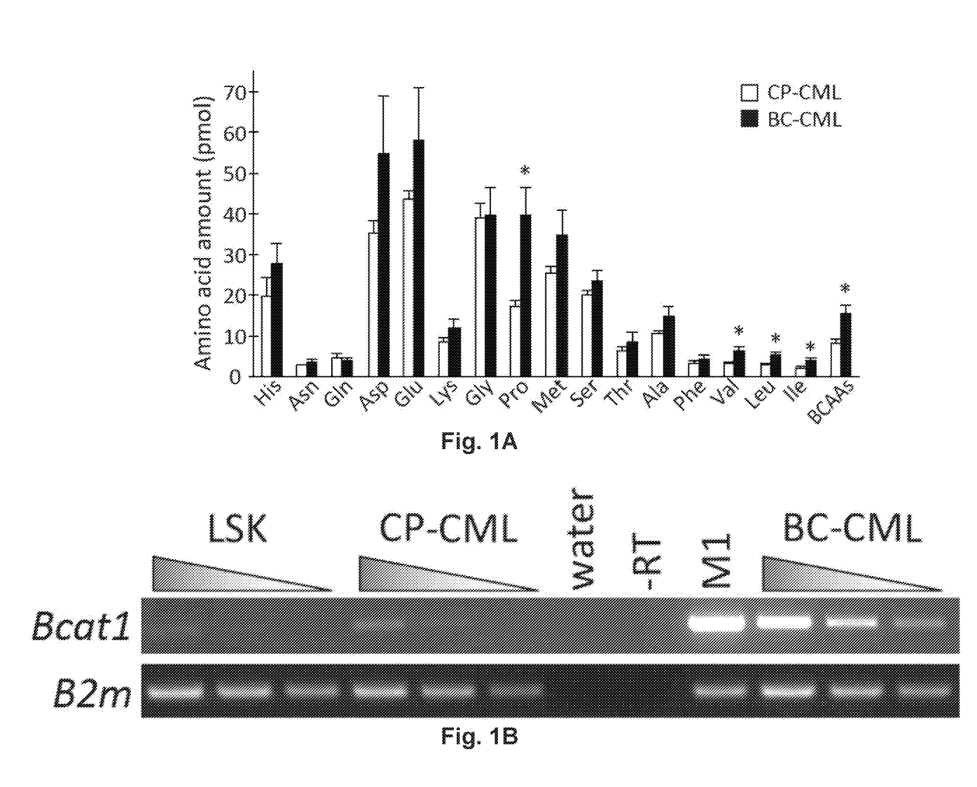

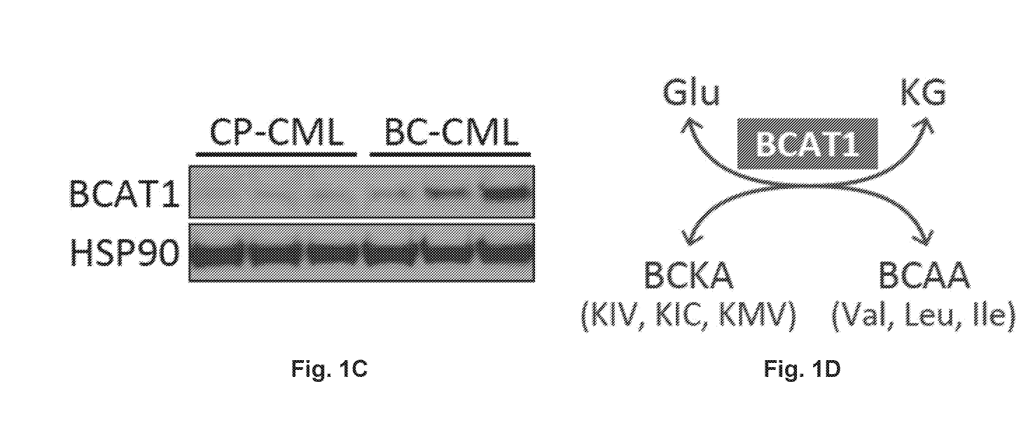

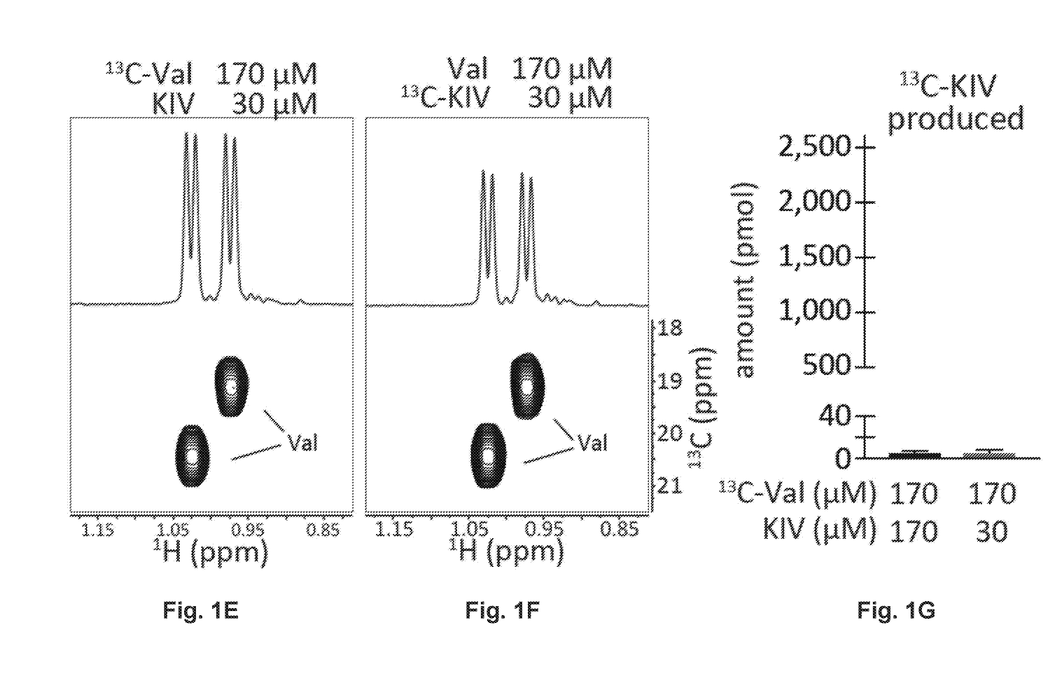

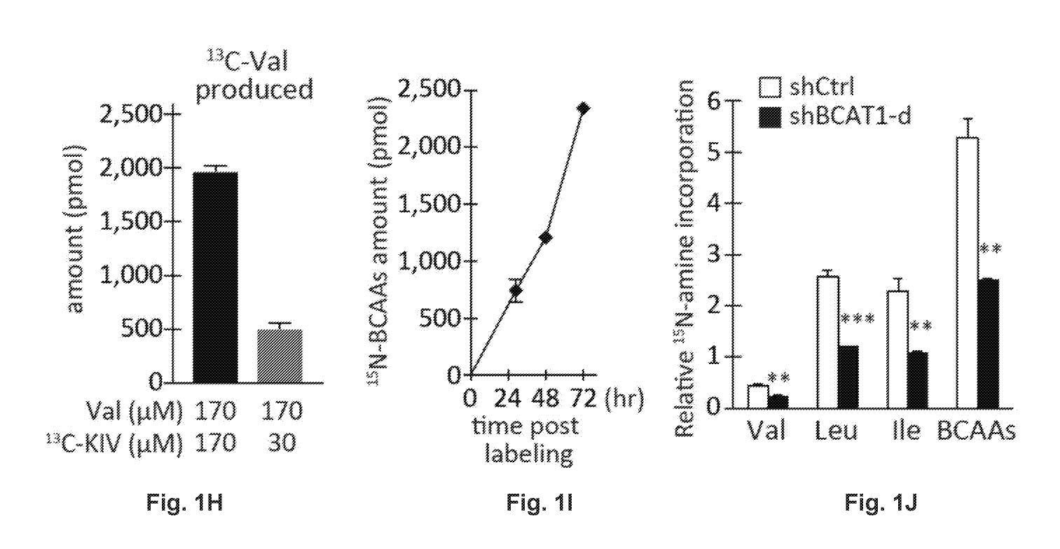

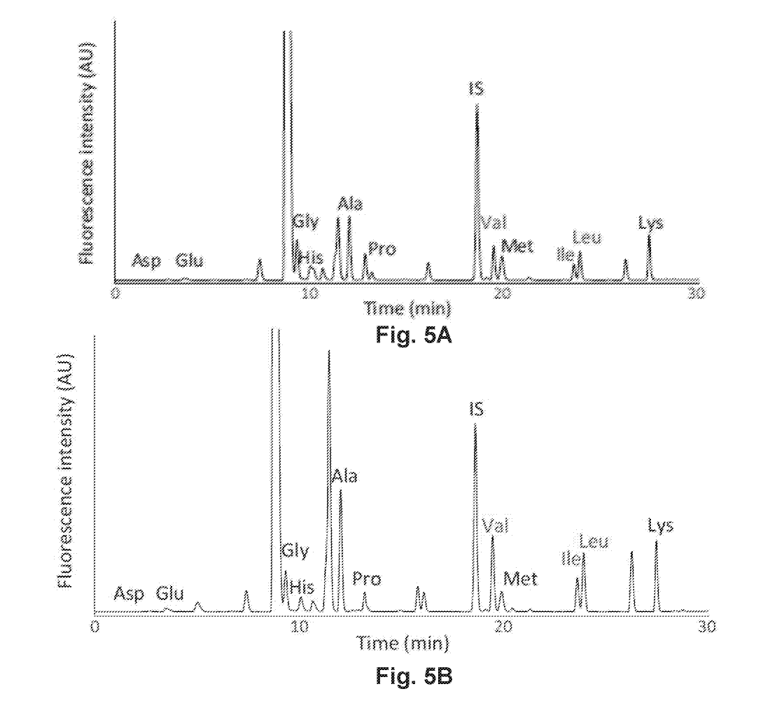

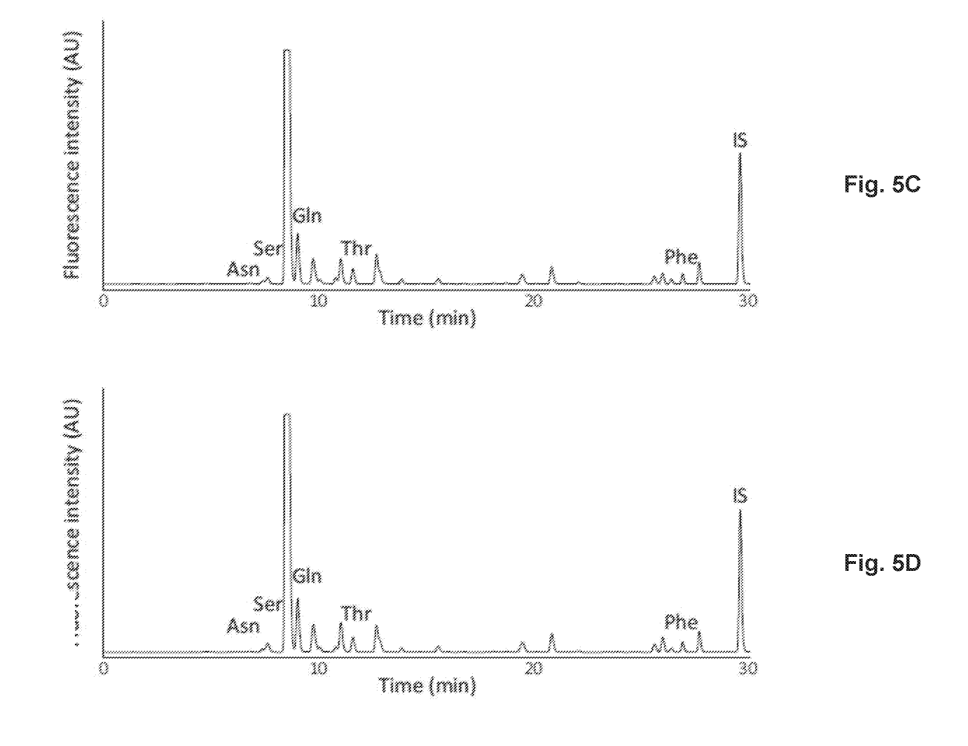

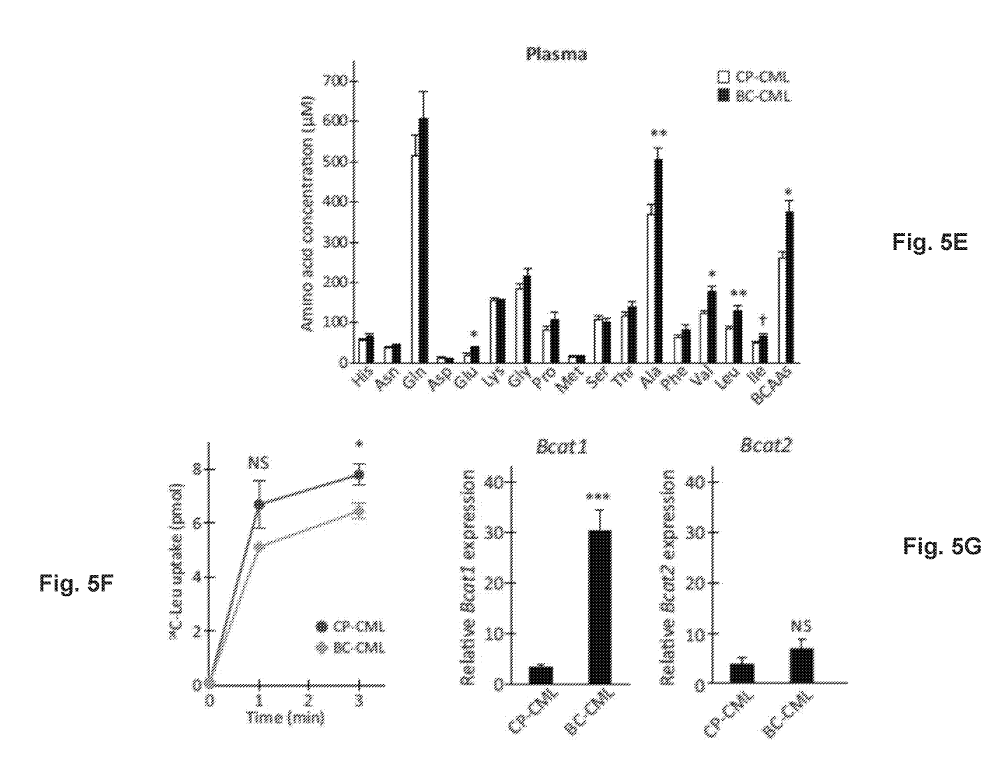

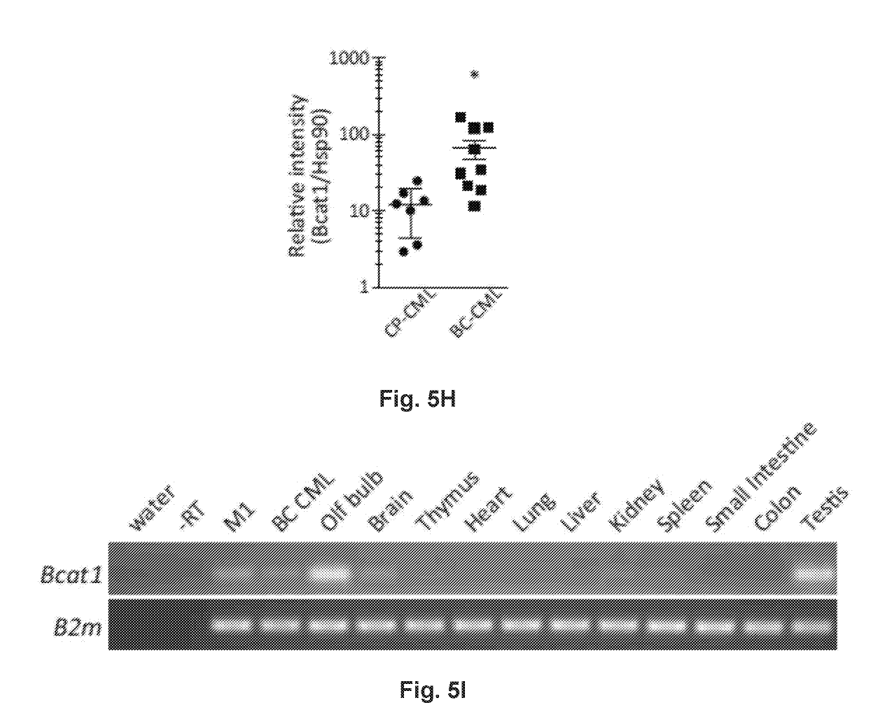

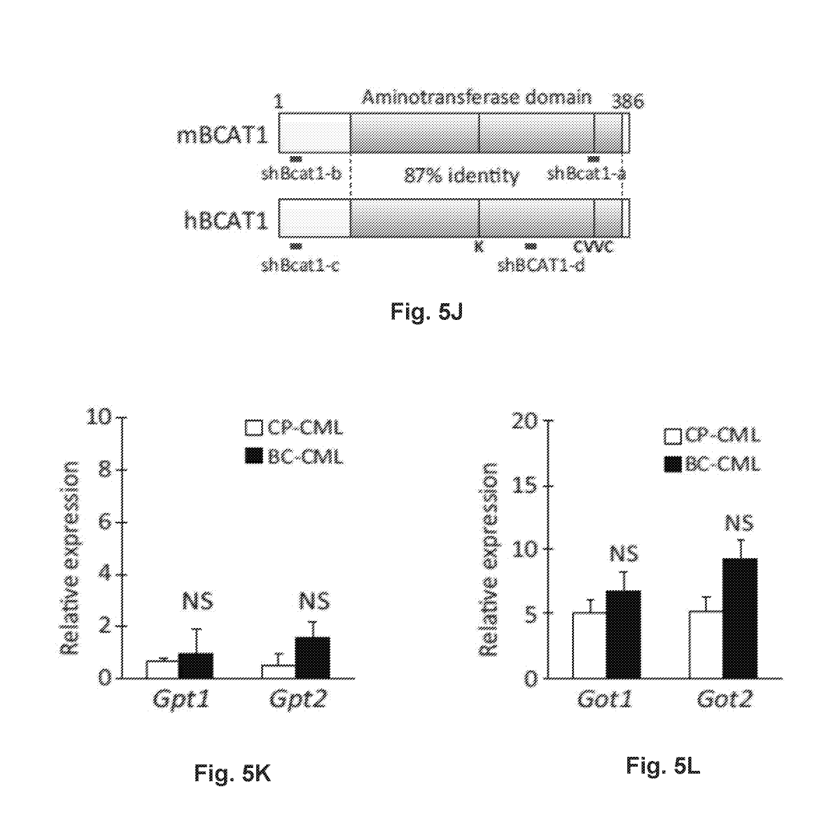

To understand the contribution of α-amino acid (AA) metabolism to the cancer progression of CML, blood AA levels were analyzed in murine models that recapitulate the chronic and blast crisis phases of human CML^{3,4}. Using amine-specific fluorescent labeling coupled with high-performance liquid chromatography, sixteen AAs were quantified in the blood plasma from leukemic mice (FIG. 5A-D). Mice bearing BC-CML showed moderate but significant elevations of plasma glutamate, alanine and the branched-chain amino acids (BCAAs; namely, valine, leucine and isoleucine) compared to CP-CML mice, indicating hyperaminoacidemia (FIG. 5E). Intracellular levels of BCAAs and proline were higher in BC-CML, whereas intracellular glutamate and alanine were comparable in the two disease phases (FIG. 1A). These results suggest that increased BCAA uptake or metabolism may contribute to CML progression. The gene expression was analyzed and no significant up-regulation of known BCAA transporters were found in BC-CML compared with CP-CML. Leucine import into BC-CML cells was not greater than into CP-CML cells (FIG. 5F), indicating that increased BCAA uptake does not explain the higher BCAA levels in BC-CML. To examine the possibility of altered intracellular BCAA metabolism, the expression of genes encoding AA metabolic enzymes was next analyzed and the branched-chain amino acid aminotransferase 1 (Bcat1) was found to be more highly expressed in BC-CML than in CP-CML at both the mRNA and protein levels (FIG. 1B-C, FIG. 5G-H). In contrast, normal hematopoietic stem/progenitor cells (HSPCs) from healthy mice had very low levels of Bcat1 expression (Lin⁻ Sca-1⁺ cKit⁺ (LSK) population; FIG. 1B), and normal tissues did not show detectable Bcat1 expression except for the brain and testis (FIG. 5I). Bcat1 encodes an evolutionarily conserved cytoplasmic aminotransferase for glutamate and BCAAs, constituting a regulatory component of cytoplasmic amino and keto acid metabolisms (FIG. 1D). Bcat2, a paralog encoding the mitochondrial BCAA aminotransferase, and alanine and aspartate aminotransferases did not show differential expression between CP- and BC-CML (FIGS. 5G-L).

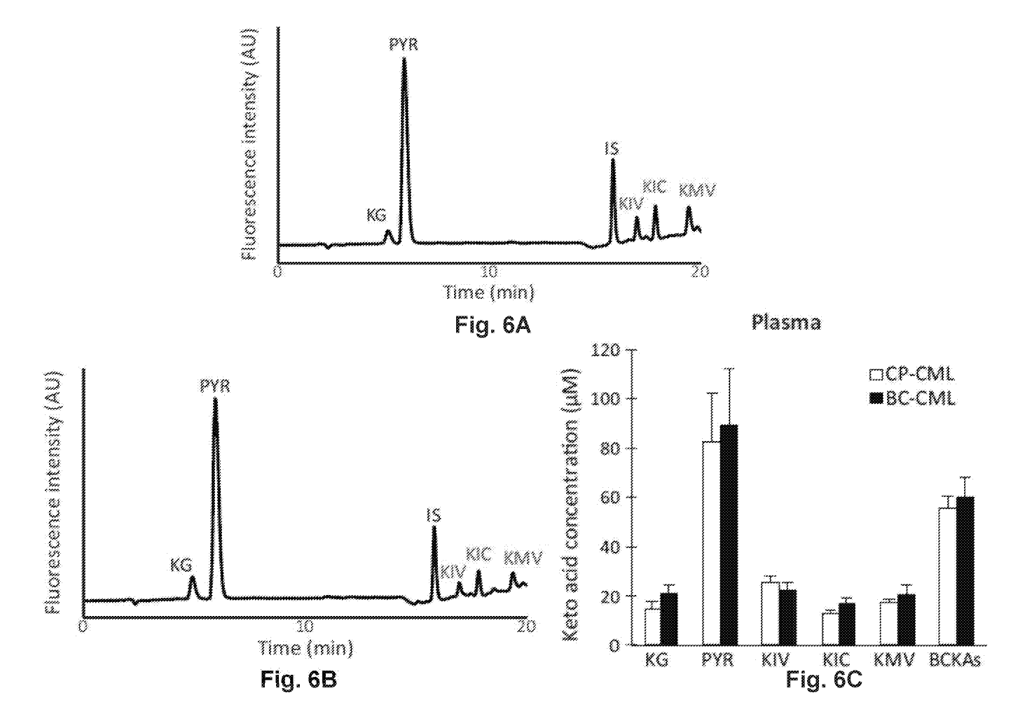

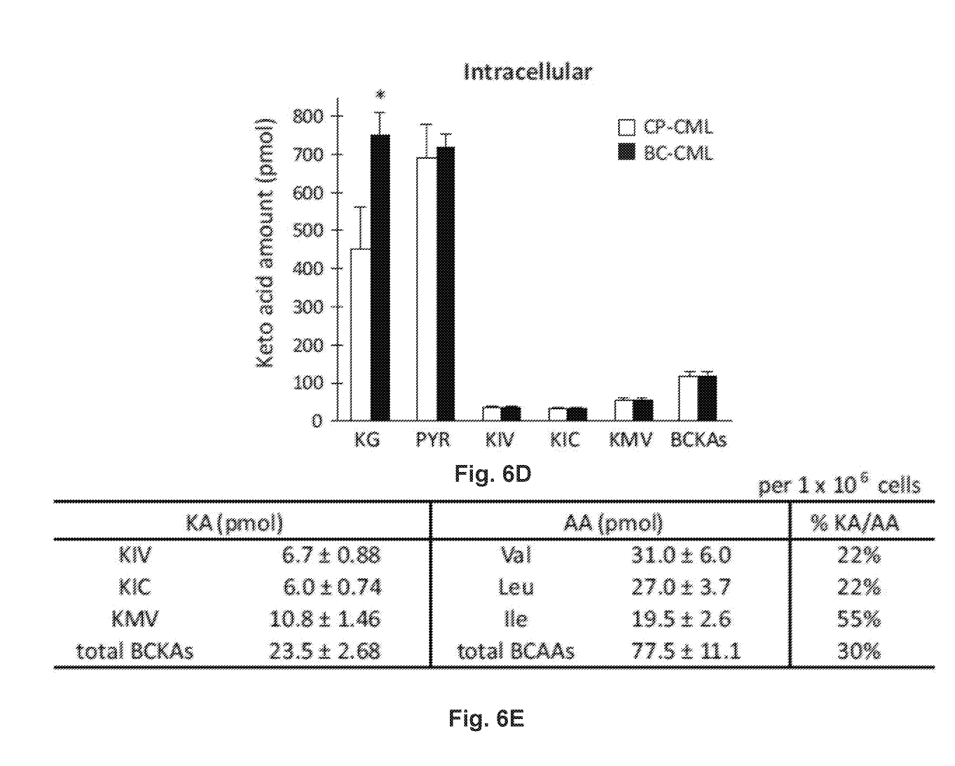

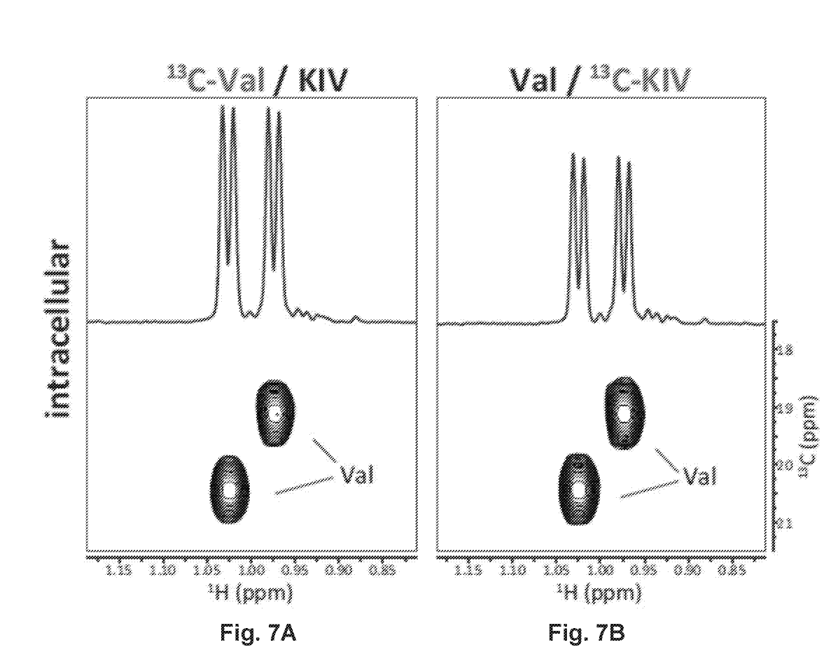

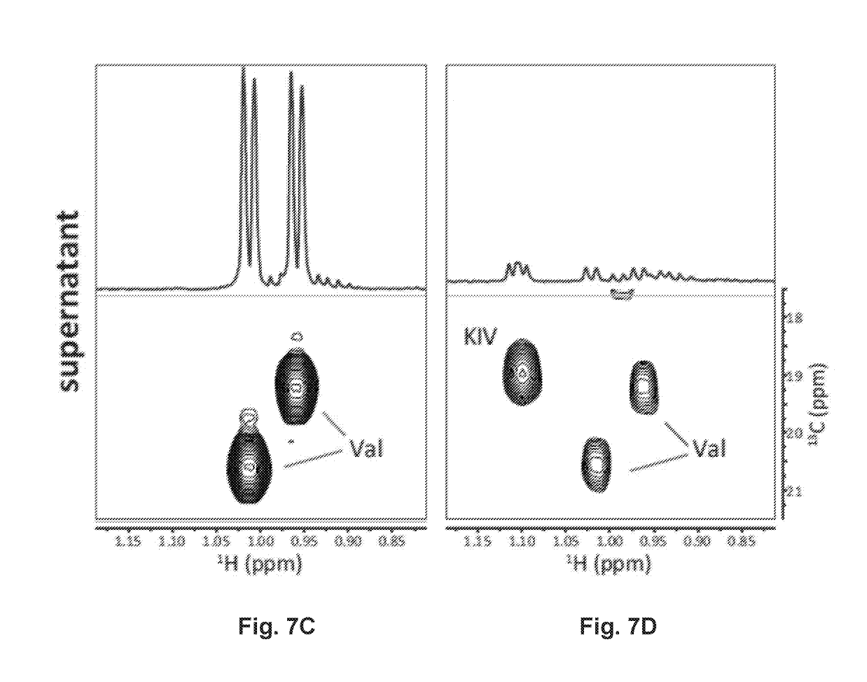

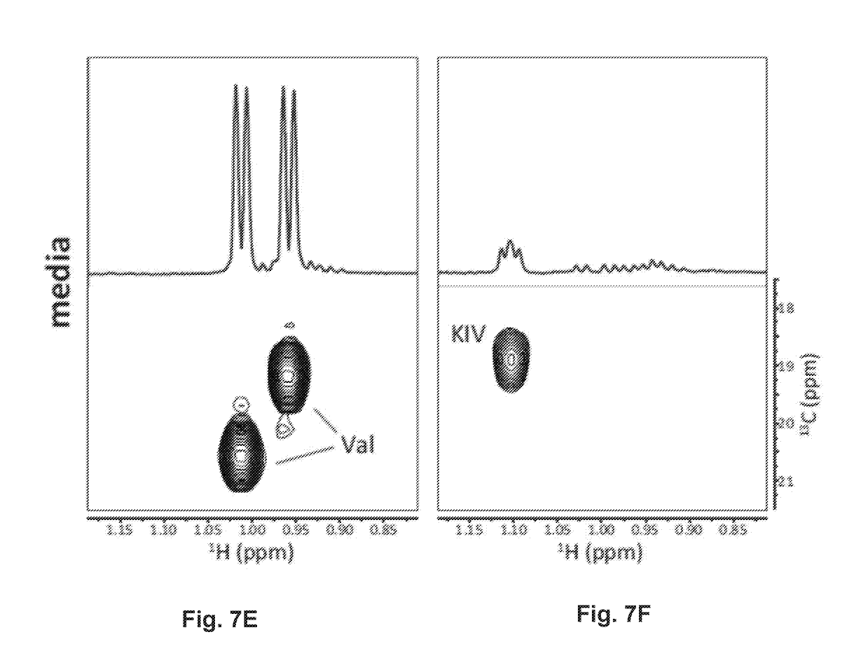

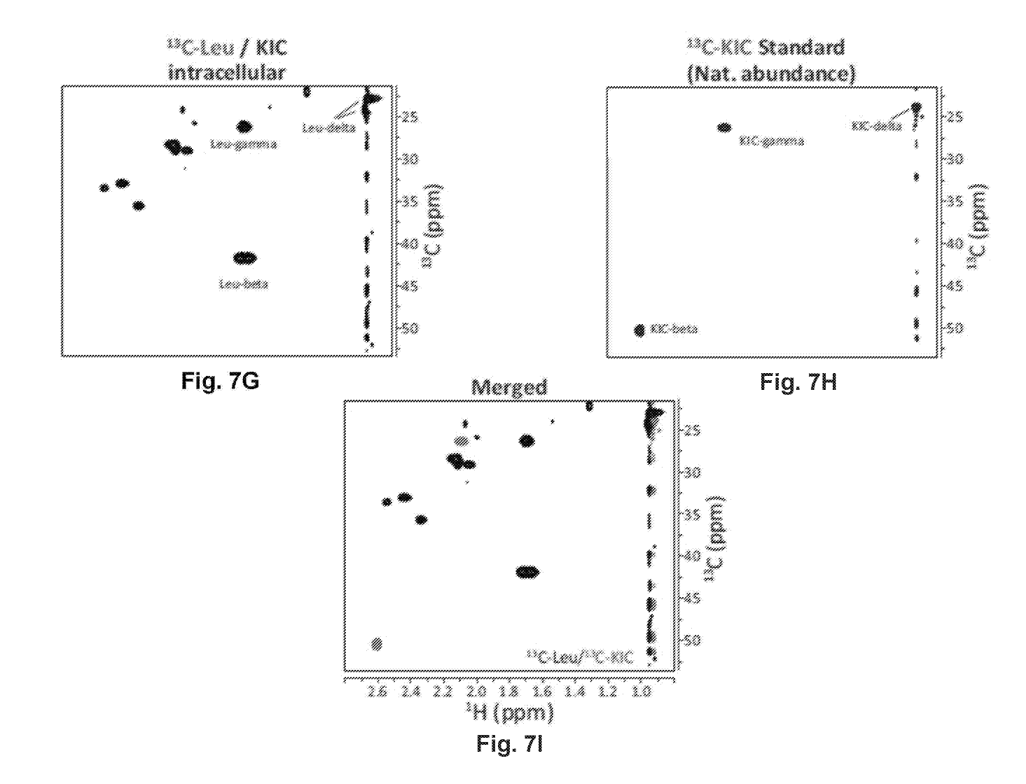

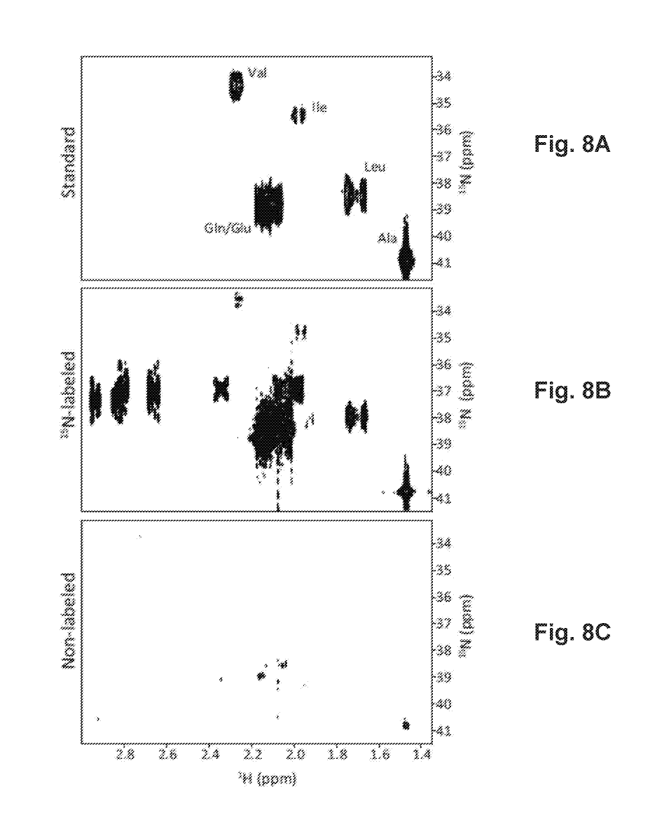

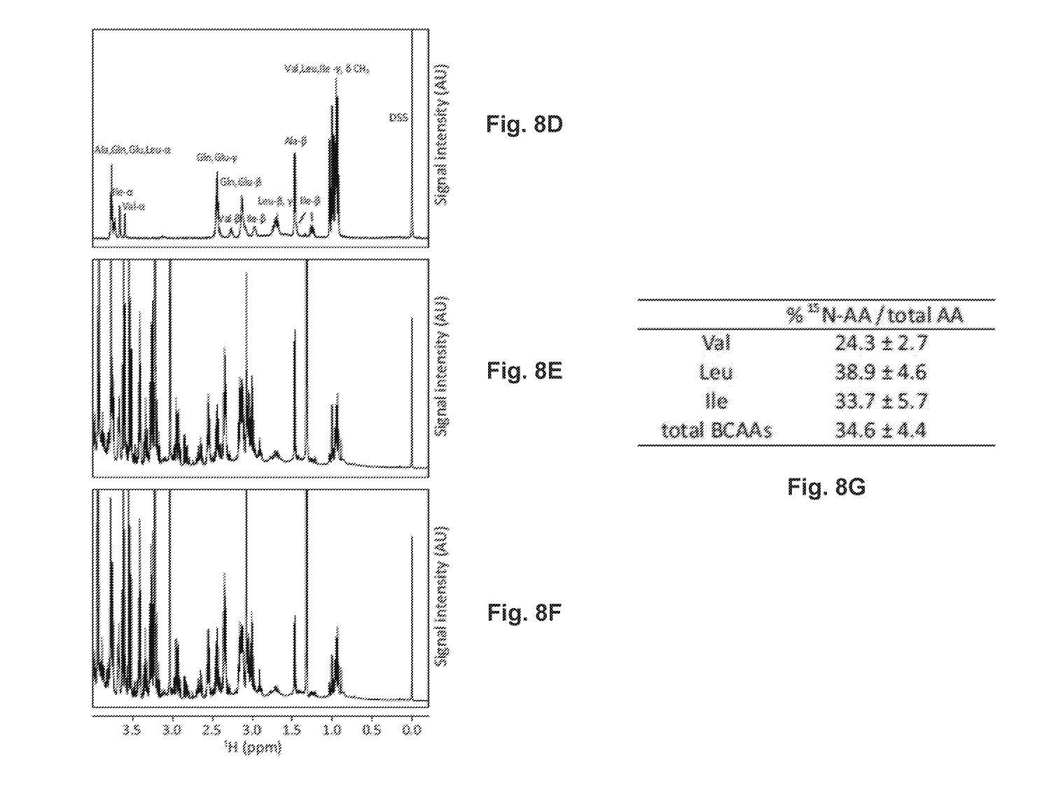

Although BCAT1 catalyzes transamination in both directions, the breakdown of BCAAs is the predominant reaction in most cell types⁶. In order for BCAT1 to generate BCAAs via the reverse reaction, the corresponding branched-chain keto acids (BCKAs), as well as glutamate, must be present as substrates. All three BCKAs, keto-isovalerate (KIV), keto-isocaproate (KIC) and keto-methylvalerate (KMV), were found present in both the blood plasma and leukemia cells (FIGS. 6A-D). In BC-CML cells, BCKAs were present at concentrations equivalent to 22-55% of the corresponding BCAAs, suggesting that intracellular BCKAs can serve as substrates for BCAA production (FIG. 6E). Next, whether BCAAs are produced through BCAT1 transamination reactions in leukemia cells by stable-isotope tracer experiments with ¹³C-valine or ¹³C-KIV was examined. Intracellular ¹³C-labeled metabolites in K562 human BC-CML cells were analyzed using one-dimensional (1D) and two-dimensional (2D) ¹H-¹³C heteronuclear single bond correlation (HSQC) analysis by high-field NMR spectroscopy (FIGS. 1E-H, FIGS. 7A-I). HSQC analysis detects only metabolites that have incorporated ¹³C. To determine whether KIV is converted to valine, cells were cultured in media supplemented with uniformly-labeled [(U)-¹³C] KIV and non-labeled valine at physiological concentrations (30 and 170 μM, respectively) and analyzed for intracellular ¹³C-metabolites. After 15 min of labeling, the generation of ¹³C-valine was clearly observed, indicating the efficient intracellular production of valine from KIV (FIGS. 1F, 1H). In contrast, ¹³C-KIV formation was barely detectable in the cells cultured with non-labeled KIV and [(U)-¹³C]-valine (FIGS. 1E, 1G). Observation of intracellular ¹³C-valine signals indicates its transport into BC-CML cells. Robust signals for ¹³C-KIV when present (FIGS. 7D, 7F) were also detected. The formation of valine from KIV, but not the breakdown of valine to KIV, was also observed when we used equal concentrations of KIV and Val in the labeling media (170 μM each; FIGS. 1G-H). KIC formation from ¹³C-leucine was not detected either (FIGS. 7G-I). These results indicate that little, if any, BCAAs are catabolized to BCKAs in leukemia cells. To further provide evidence for the intracellular BCAA production through transamination, alternative labeling experiments were performed to track the fate of the amine group of glutamate. K562 cells were cultured with ¹⁵N-amine-labeled glutamine, which is metabolized to ¹⁵N-amine-glutamate by glutaminase upon cellular intake, and the subsequent labeling of BCAAs analyzed via ¹H NMR and ¹H-¹⁵N heteronuclear multiple bond correlation (HMBC) analysis. HMBC analysis detects only metabolites that have incorporated ¹⁵N, whereas ¹H NMR detects any compounds containing protons (FIGS. 8A-F). At 29-72 h post-labeling, ¹⁵N-amine-labeled BCAAs were detected, indicating transamination from glutamine to BCAAs (FIG. 1I). By 72 h, the ¹⁵N-amine-labeled BCAAs had accumulated to fractional abundances ranging from 24 to 39% (FIG. 8G), indicating a significant contribution of transamination to the intracellular BCAA pool. Lentiviral BCAT1 knockdown resulted in greater than a 50% decrease in the amount of intracellular BCAAs produced (FIG. 1J). These data demonstrate that BCKA transamination by BCAT1 contributes to the BCAA pool in leukemia cells.

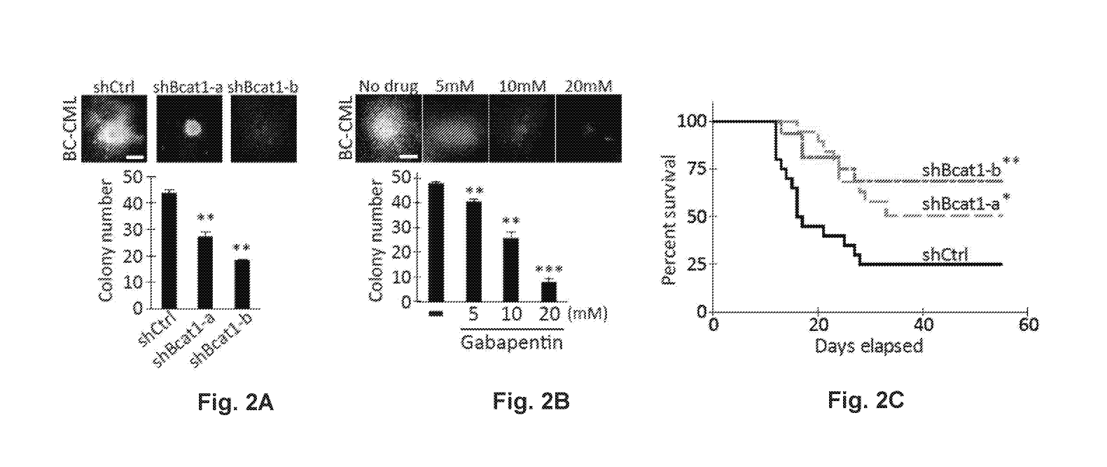

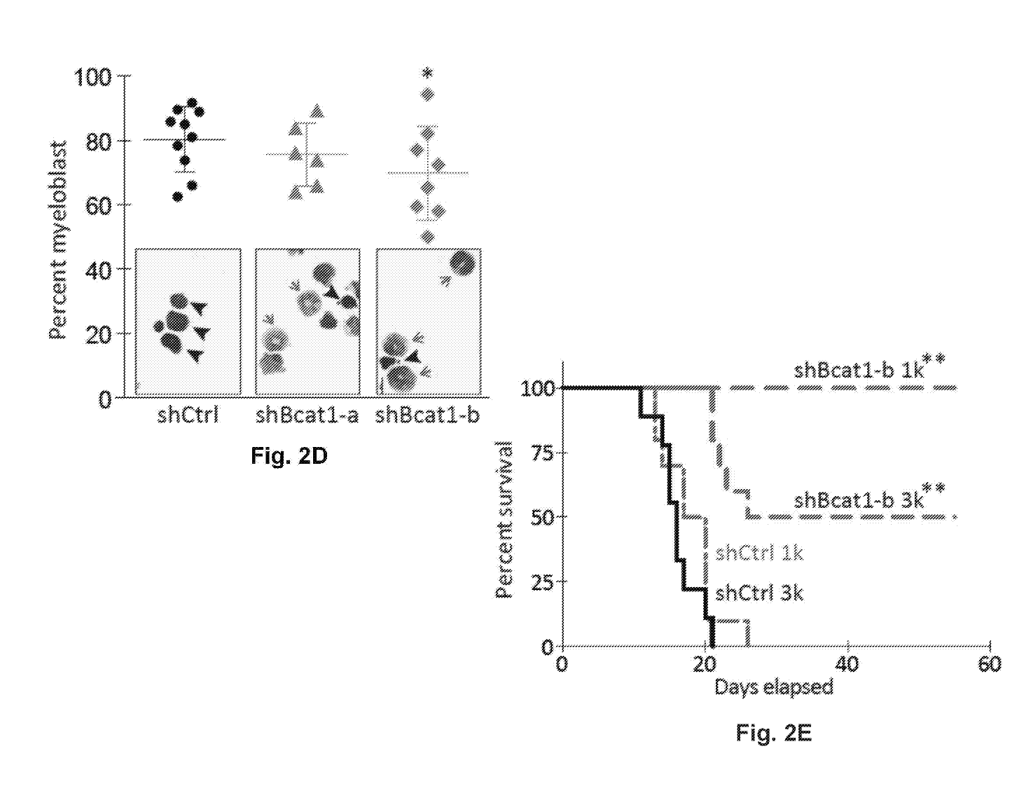

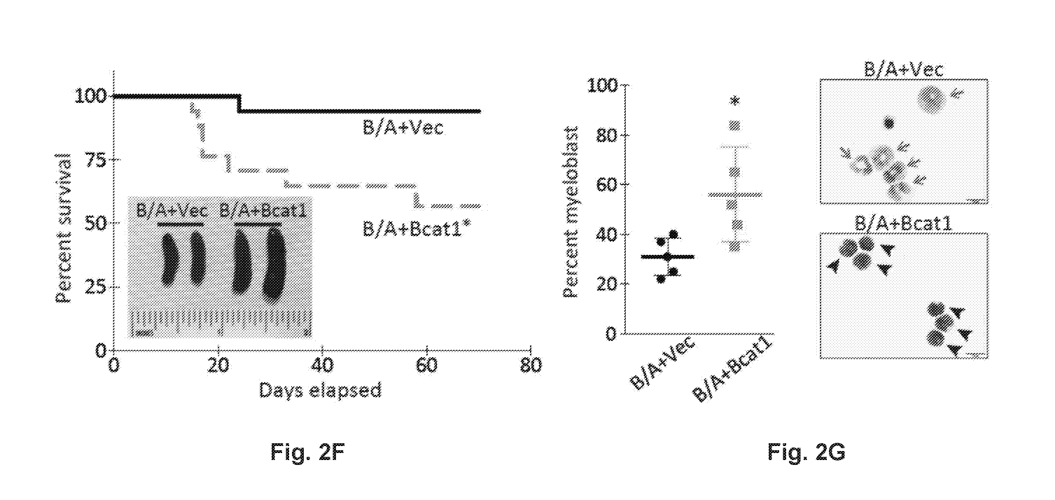

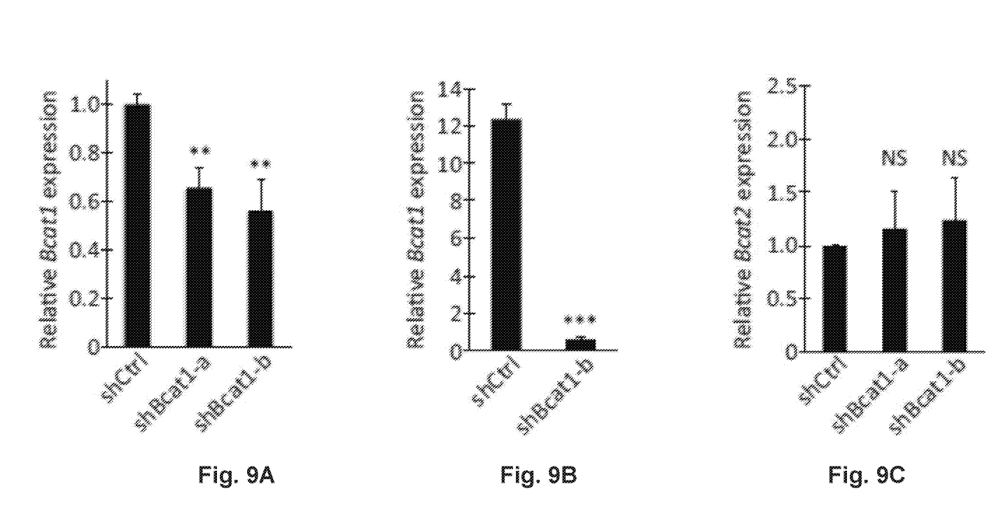

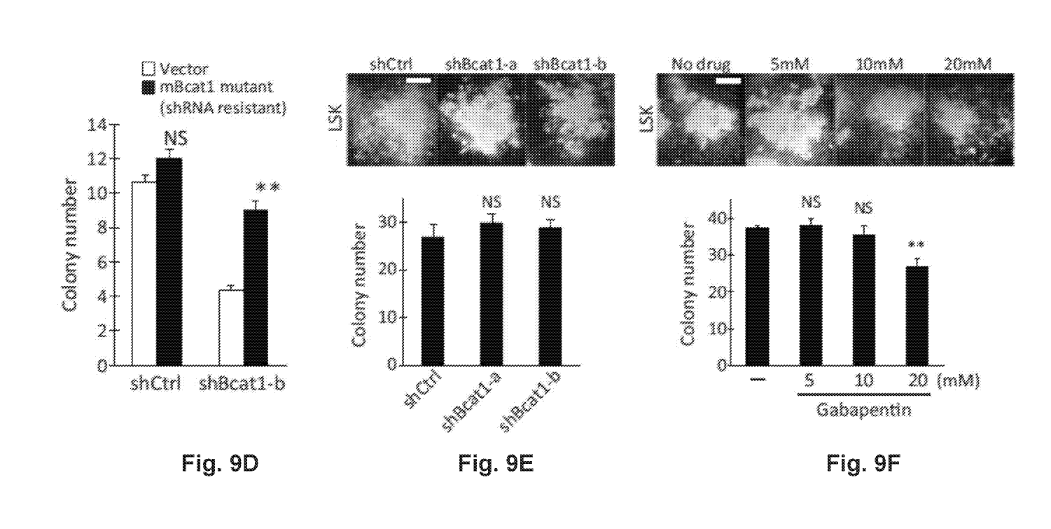

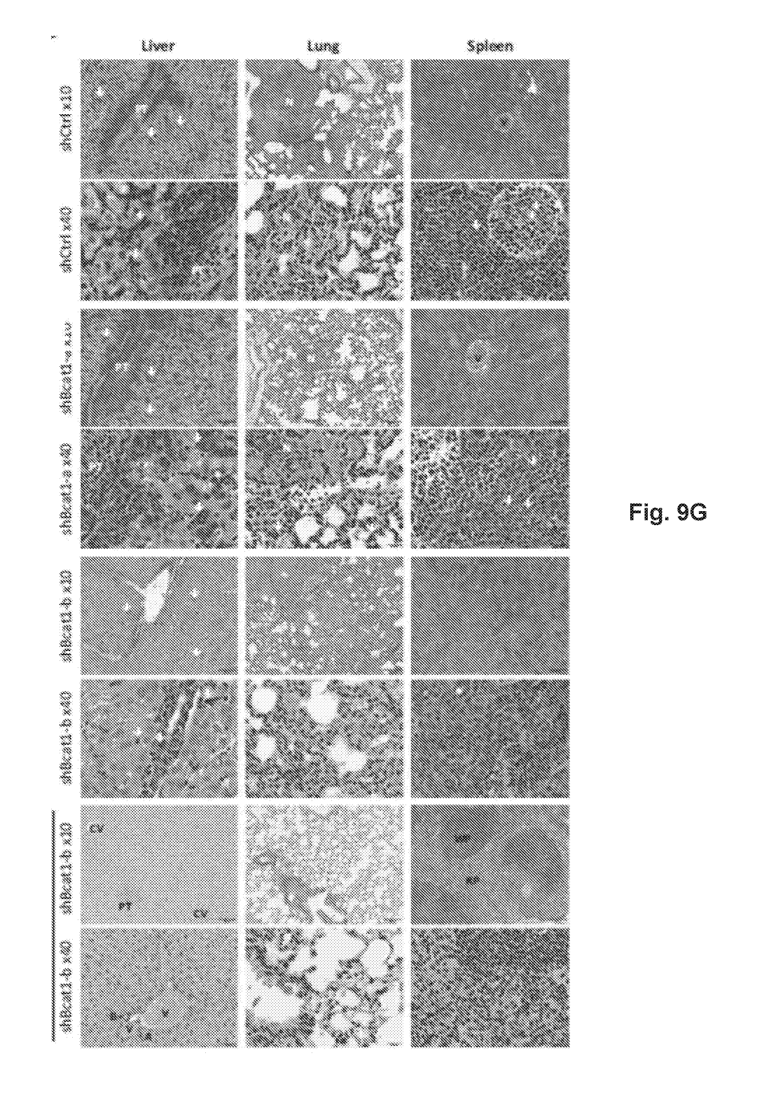

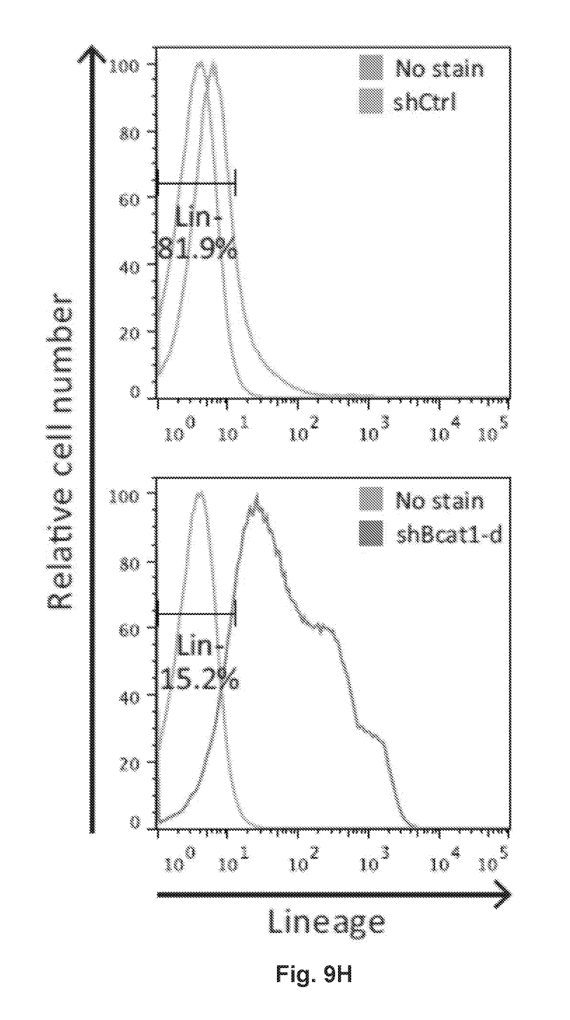

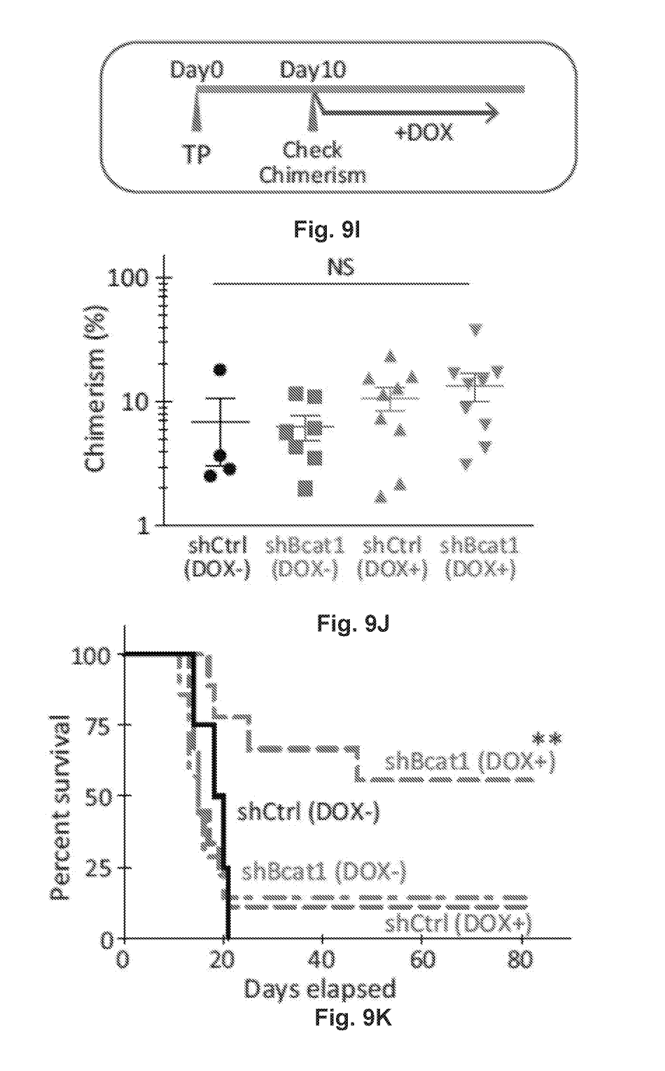

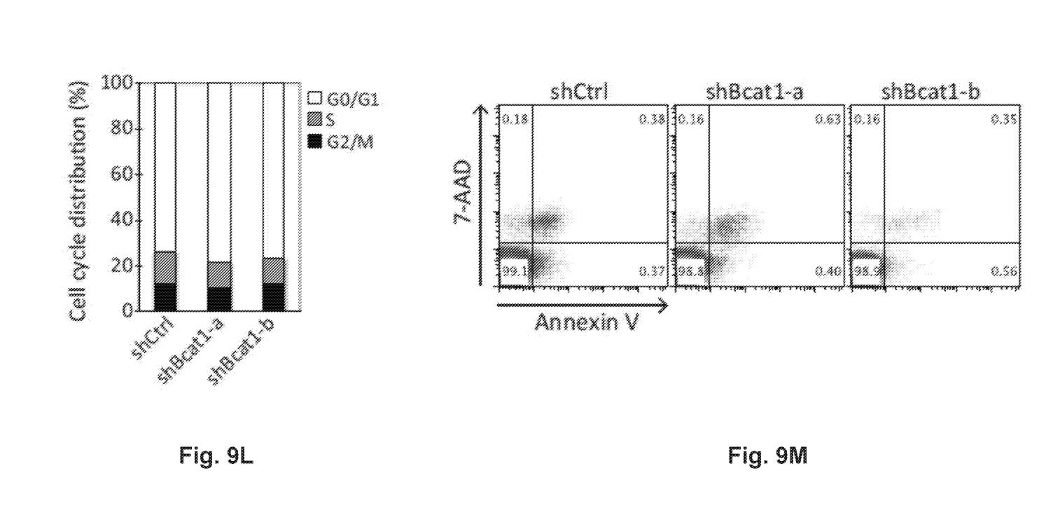

Given that Bcat1 is highly expressed and augments intracellular BCAAs in BC-CML, Bcat1 may functionally contribute to the acute properties of BC-CML. To test this possibility, Bcat1 expression was inhibited using a short hairpin RNA (shRNA)-mediated gene knockdown approach. The immature lineage-negative (Lin⁻) cells were sorted from primary BC-CML samples, a population that contains the leukemia-initiating cells of this cancer, and introduced two independent retroviral shRNA constructs (FIG. 5J; shBcat1-a and shBcat1-b). Both constructs inhibited Bcat1 expression in BC-CML compared with a non-targeting negative control shRNA (shCtrl) (FIGS. 9A-C). Bcat1 knockdown resulted in significantly smaller colonies and a 40-60% reduction in the colony-forming ability relative to a control (FIG. 2A). The co-introduction of a shRNA-resistant Bcat1 cDNA rescued the reduced clonogenic potential (FIG. 9D). As an alternative approach to gene knockdown, BC-CML cells were treated with gabapentin (Gbp), a chemical inhibitor of BCAT1. Gbp is a structural analog of leucine and specifically and competitively inhibit the transaminase activity of BCAT1 but not that of BCAT2⁷. BC-CML cells plated with Gbp formed smaller colonies and showed a dose-dependent impairment in clonogenic growth (FIG. 2B). In contrast, normal HSPCs were only minimally affected by gene knockdown or Gbp treatment (FIGS. 9E-F). These data show that BCAT1 inhibition may selectively impair the propagation of leukemia without affecting normal hematopoiesis.

To examine whether Bcat1 loss affects the propagation of BC-CML in vivo, Lin⁻ cells expressing shBcat1 were transplanted into conditioned recipient mice. Whereas 75% of the recipients transplanted with control cells succumbed to the disease within 30 days, only 47% (shBcat1-a) and 31% (shBcat1-b) of the mice transplanted with Bcat1-knockdown cells developed the disease, and more than half of these mice survived even when followed out to 60 days (FIG. 2C). Among the mice that developed disease with Bcat1 knockdown, most had leukemia that was characterized by differentiated granulocytes and lower levels of immature myeloblasts (FIG. 2D, FIG. 8G). They also displayed a lower frequency of immature Lin⁻ cells than control leukemia (FIG. 9H), indicating that the loss of Bcat1 induced differentiation and impaired the leukemia-initiating cell activity. Consistent with these phenotypes, serial transplantation of the leukemia cells revealed that while all the control leukemia propagated the disease, none of the mice transplanted with Bcat1-knockdown leukemia cells succumbed to the disease (1k shown in FIG. 2E). In addition, a doxycycline (Dox)-inducible Bcat1 knockdown system (i-shBcat1) was established and the impact of Bcat1 loss on the disease maintenance examined. Ten days post-transplantation with BC-CML cells infected with i-shBcat1, leukemic engraftment was assessed in each recipient, and Dox treatment was initiated (FIGS. 9I-J). While almost all the mice that were transplanted with control cells and the non-Dox-treated mice developed leukemia, more than half of the Dox-treated i-shBcat1 mice remained disease-free (FIG. 9K), indicating that Bcat1 is required for the continuous propagation of BC-CML. At the cellular level, neither enhanced apoptosis nor a decrease in actively cycling cells by Bcat1 knockdown were observed (FIGS. 9L-M). These results demonstrate that Bcat1 is critical for the sustained growth and maintenance of leukemia-initiating cells in BC-CML.

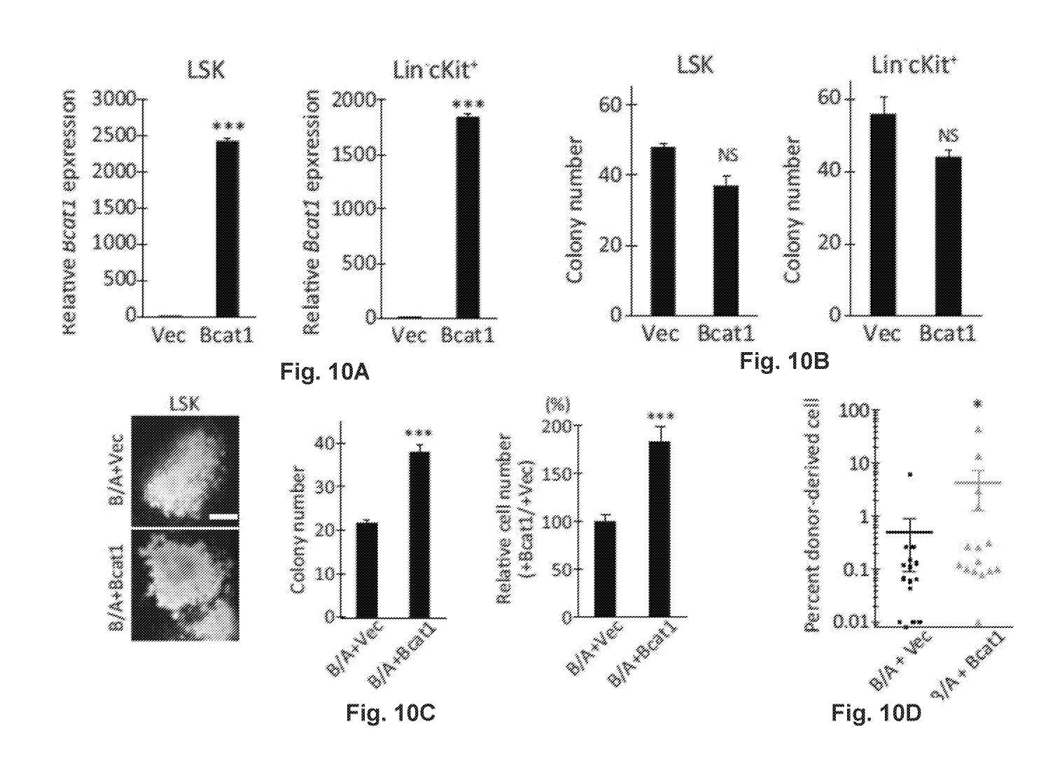

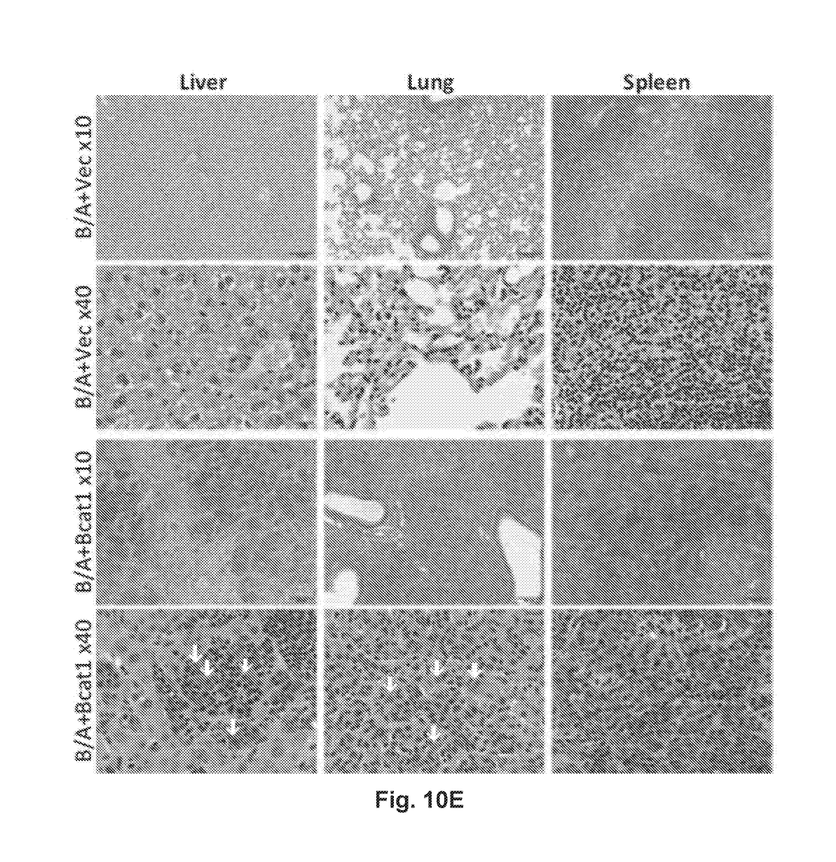

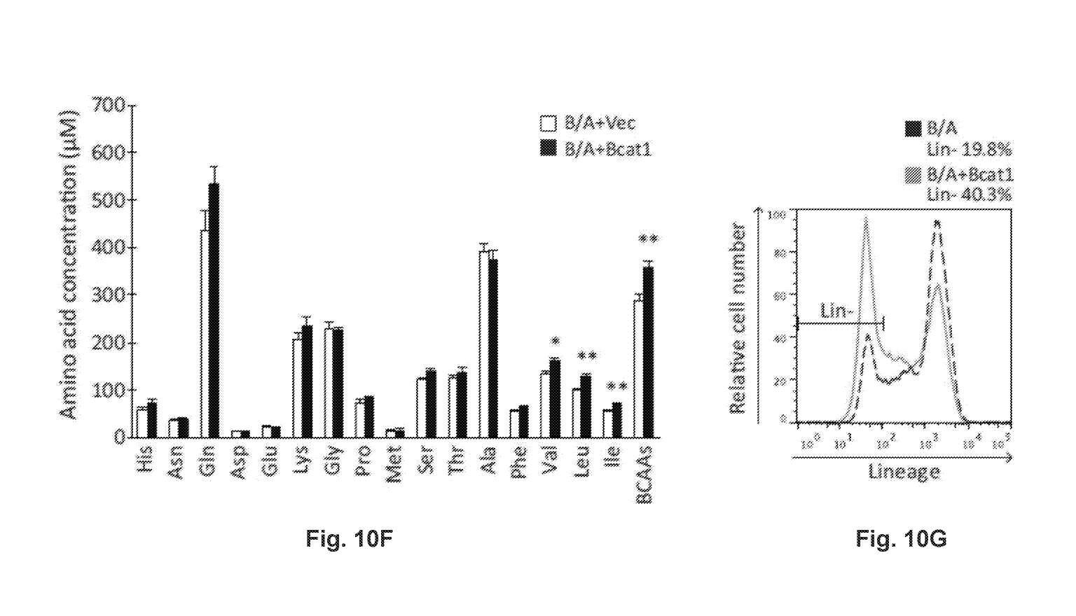

It was next examined whether the enforced expression of Bcat1 could drive blastic transformation in hematopoietic cells. Although a significant increase in Bcat1 expression was observed compared with the vector control, Bcat1 expression alone did not enhance the colony-forming ability of either LSK or Lin⁻ c-Kit⁺ hematopoietic cells isolated from normal bone marrow (FIGS. 10A-B). To determine whether BCR-ABL1 cooperates with Bcat1 overexpression to confer an aggressive growth phenotype, normal HSPCs were transduced with Bcat1 and BCR-ABL1. Compared with the vector control, the combinatorial expression promoted clonogenic growth in vitro (FIG. 10C), and the transplantation of the cells led to significantly elevated leukemia burdens (FIG. 10D-E), splenomegaly and increased mortality in the recipient mice (FIG. 2F), with a concomitant increase in plasma BCAA levels (FIG. 10F). Accordingly, leukemia that developed in response to Bcat1 overexpression exhibited a highly immature myeloblastic morphology compared to the control (FIG. 2G, FIG. 10G). These data indicate that activated Bcat1 mediates the blastic transformation of CP-CML cells.

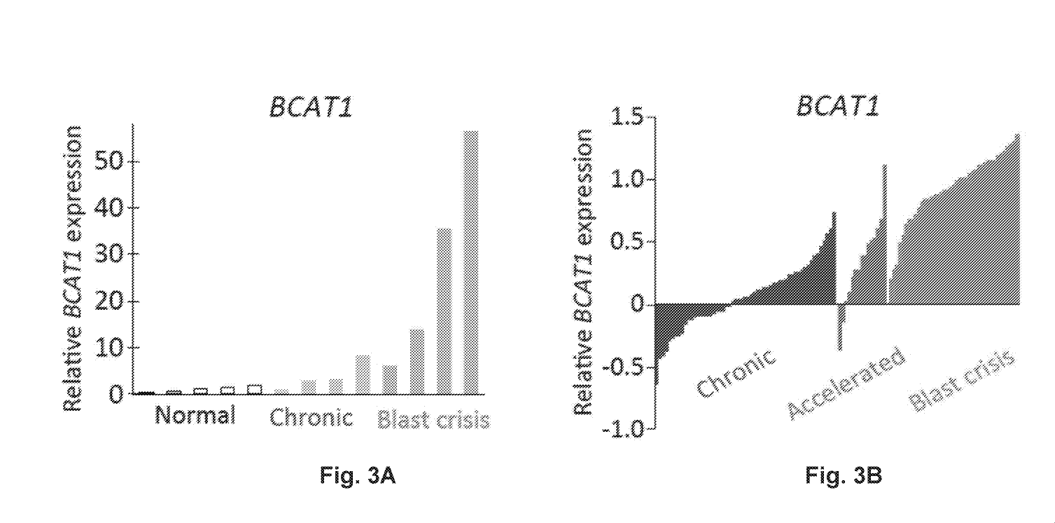

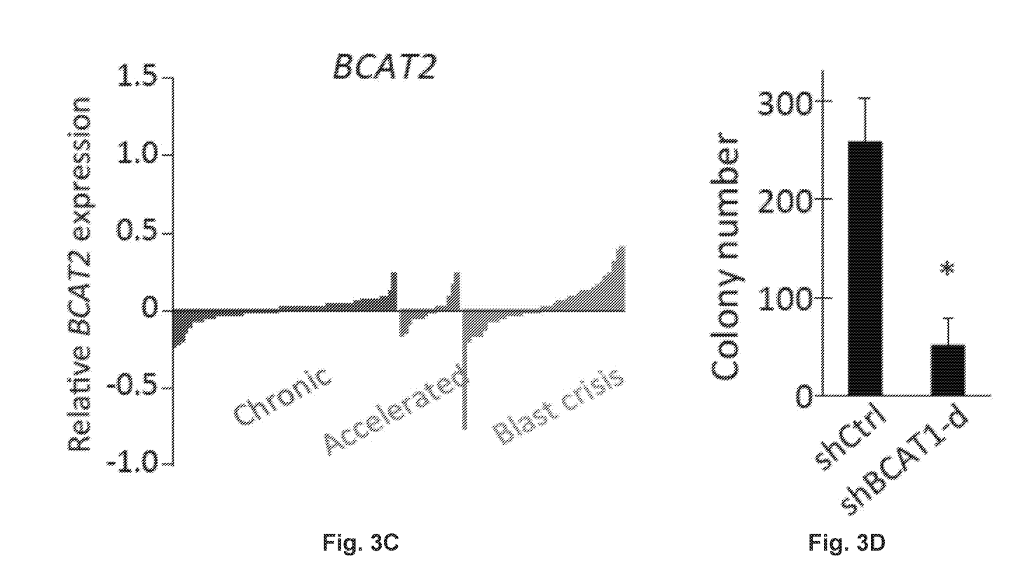

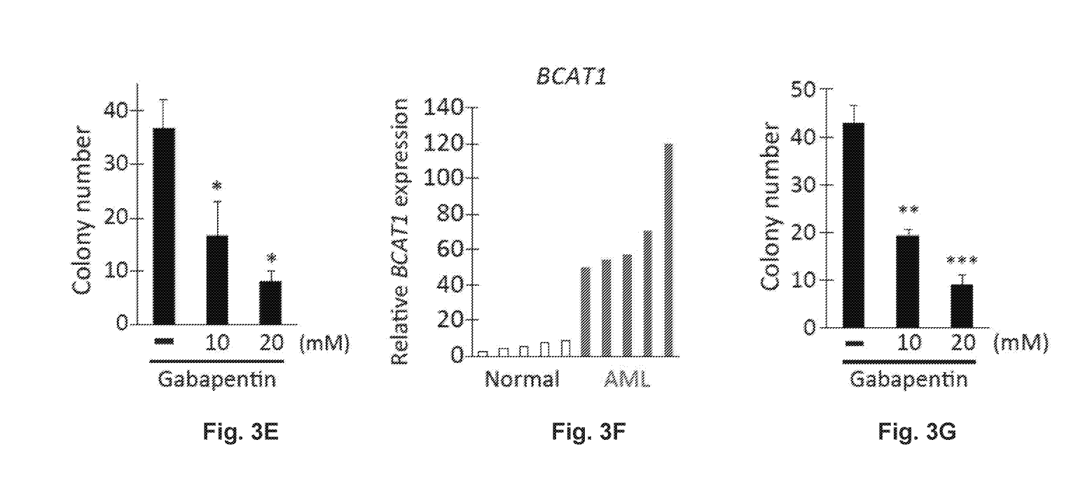

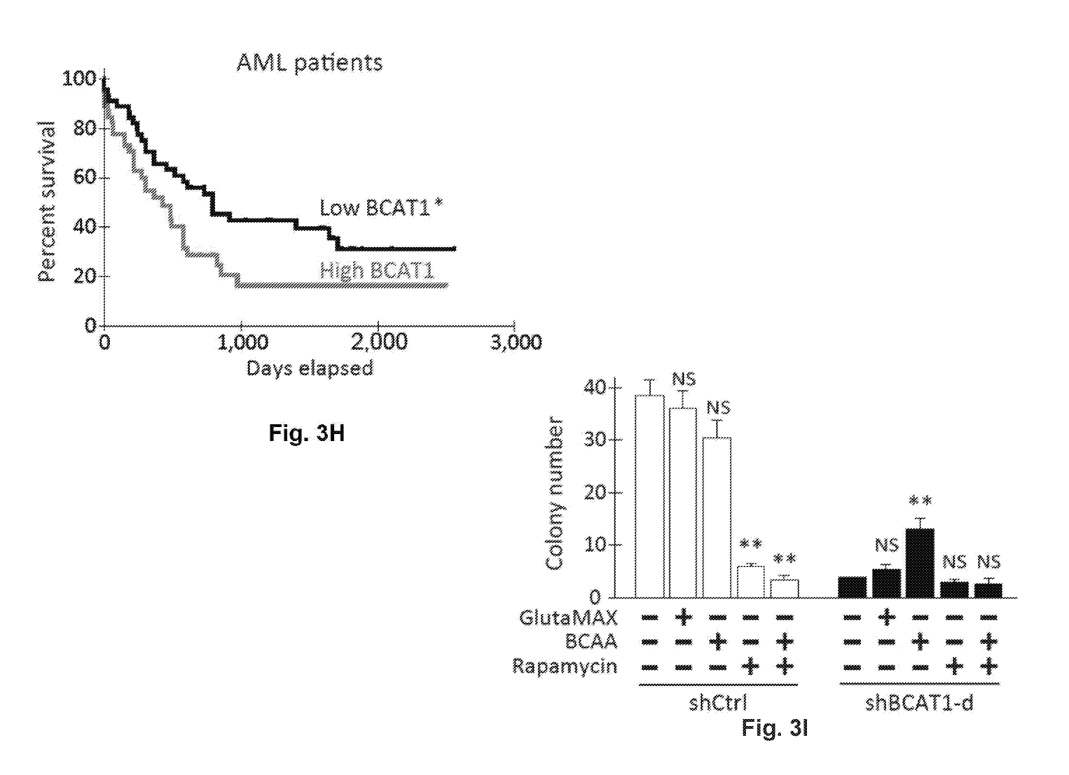

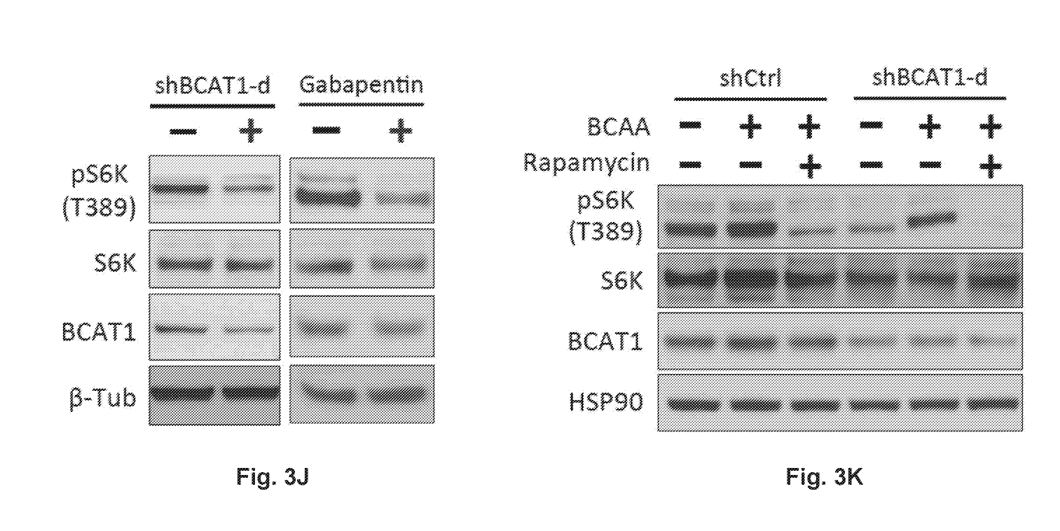

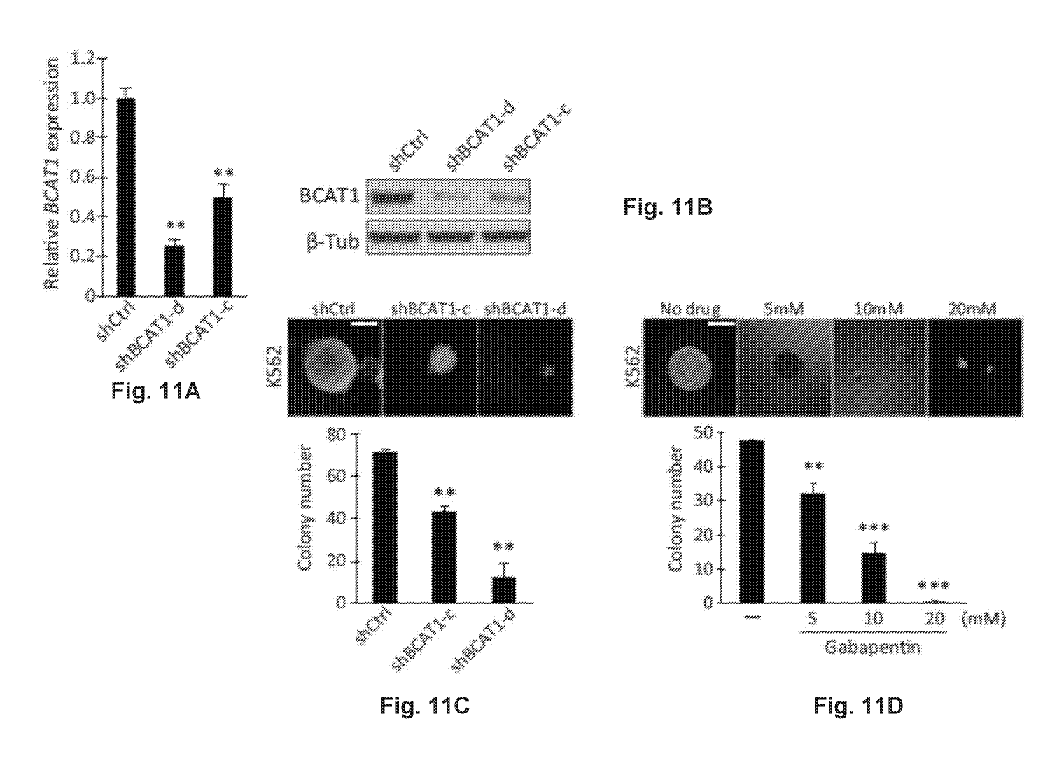

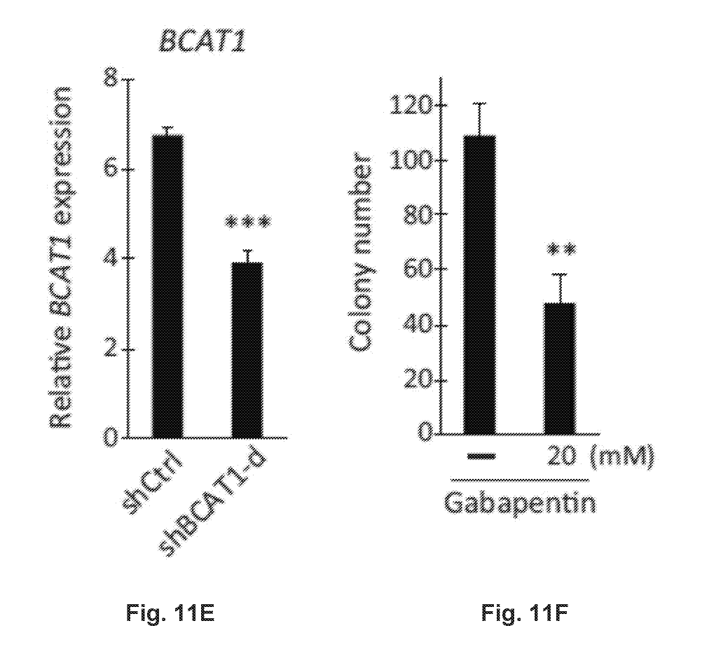

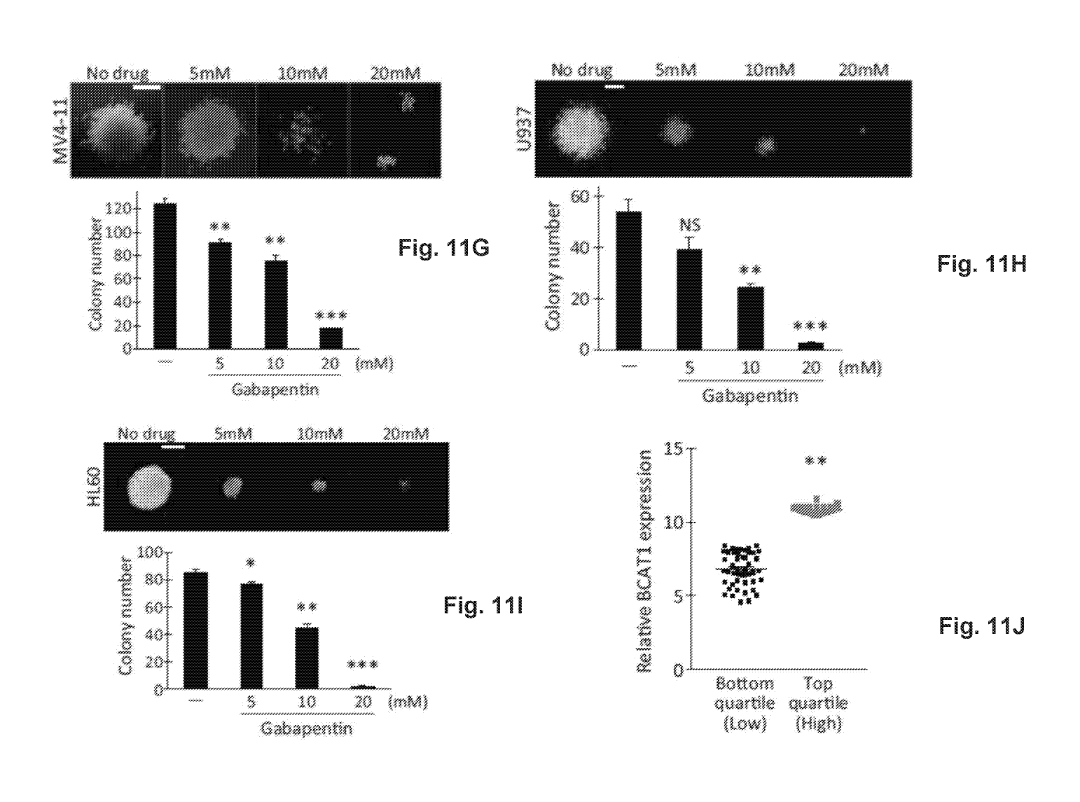

These results demonstrate that Bcat1 is essential for the development of BC-CML in mice, while normal bone marrow HSPCs show a very limited dependence on this metabolic enzyme. To investigate the contribution of BCAT1 to human leukemia, a panel of 13 peripheral blood samples from healthy and leukemic subjects was examined and it was found that human BCAT1 expression was higher in BC-CML than in either normal or CP-CML cells (FIG. 3A). To determine whether this expression pattern reflects a general trend in human CML, BCAT1 levels in a GEO dataset of 113 CML patient samples⁸ were analyzed. This focused analysis revealed a significant elevation in BCAT1 expression as the disease progresses from the chronic to the accelerated phase and then to the blast crisis phase (FIG. 3B). On average, BCAT1 expression was 15-fold higher in BC-CML than in CP-CML. No significant changes in BCAT2 expression were found, which is consistent with the results from the mouse models (FIG. 3C, FIG. 5G). These data indicate that activation of BCAT1 is a shared characteristic in the progression of human CML. Lentiviral BCAT1 knockdown or Gbp treatment markedly inhibited the colony-forming ability of K562 human BC-CML (FIG. 11A-D) and patient-derived primary leukemia cells (FIGS. 3D-E, FIG. 11E-F). Interestingly, BCAT1 activation in primary human acute myeloid leukemia was observed as well (AML; FIG. 3F), and Gbp effectively inhibited the clonal growth of human AML cell lines and primary de novo AML cells (FIG. 3G, FIG. 11G-I). Moreover, BCAT1 expression levels predict disease outcome in patient cohorts. Cases from the TCCA AML dataset were divided into quartiles based on BCAT1 expression levels (FIG. 11J), and it was found that the median survival time was 46% shorter in the BCAT1-high group (427 vs. 792 days; FIG. 3H). These results demonstrate an essential role for BCAT1 in the pathogenesis of a wide array of human myeloid malignancies.

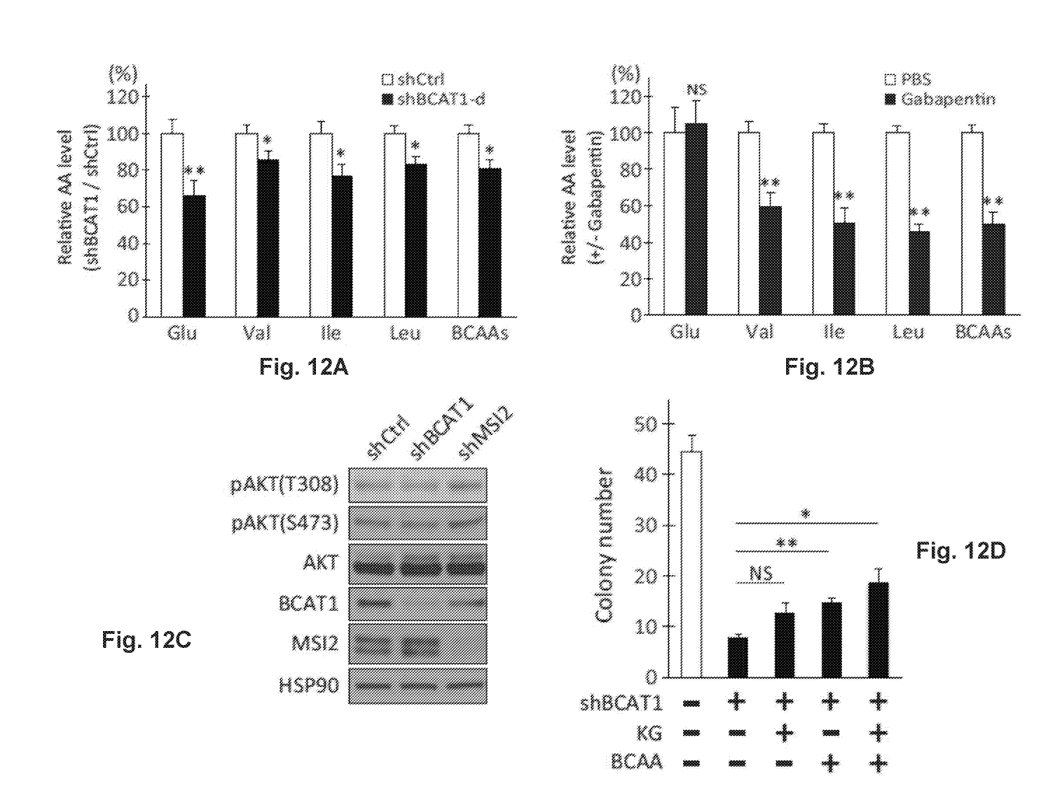

To understand how the BCAT1-driven change in metabolism promotes leukemia growth, intracellular AA concentrations were analyzed upon BCAT1 inhibition and it was found that all three BCAAs were significantly reduced by shBCAT1 or Gbp treatment compared with the controls (FIGS. 12A-B). Interestingly, the addition of BCAAs, but not alanyl-glutamine (GlutaMax), functionally suppressed the reduction of colony-forming ability caused by BCAT1 knockdown (FIG. 3I), suggesting that BCAT1 enhances clonogenic growth through BCAA production via BCKA reamination. BCAAs, particularly leucine, activate the mTORC1 pathway via cytosolic leucine sensor proteins, which integrate multiple signals from nutrient sensing and growth factor stimuli to promote cell growth⁹⁻¹². Thus, it was examined whether reduced BCAA production by BCAT1 inhibition results in the attenuation of the mTORC1 signal. Indeed, BCAT1 blockade by either shRNA or Gbp treatment significantly reduced the phosphorylation of S6 kinase (pS6K), a downstream target of mTORC1 kinase (FIG. 3J), suggesting BCAT1 activation of the mTORC1 pathway. No apparent changes were observed in the levels of phosphorylated AKT upon BCAT1 inhibition, suggesting a predominant contribution of BCAA nutrient signals to the activation of mTORC1 (FIG. 12C). Consistently, the mTORC1 inhibitor rapamycin reversed the BCAA-induced suppression of colony formation (FIG. 3I) and the BCAA-induced increase in pS6K (FIG. 3K).

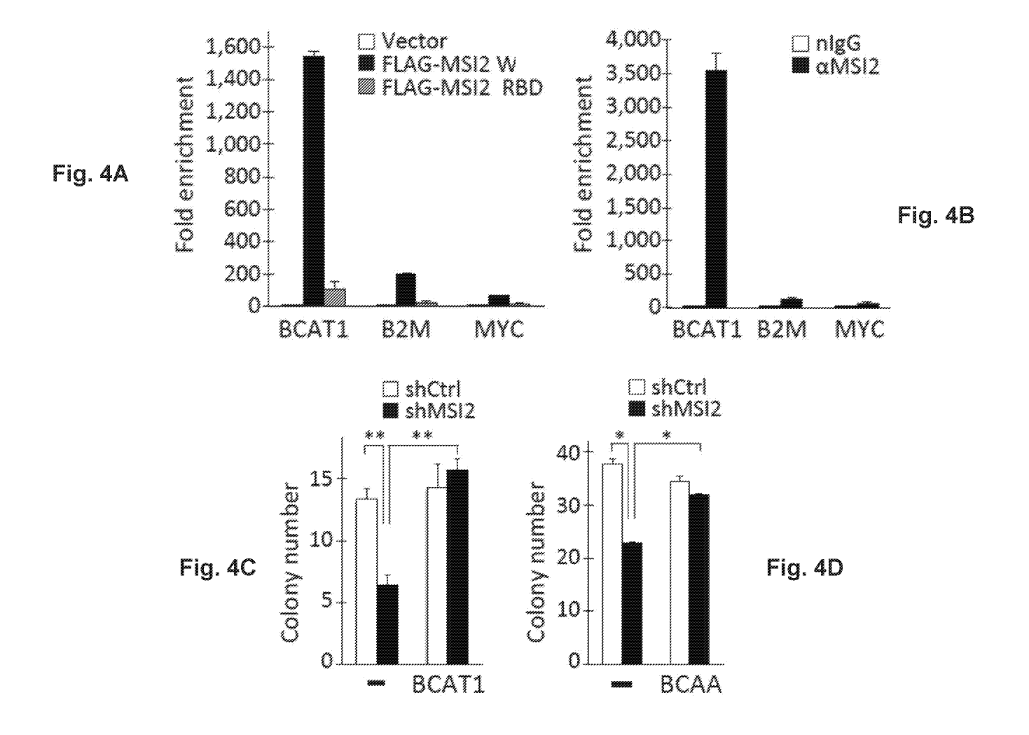

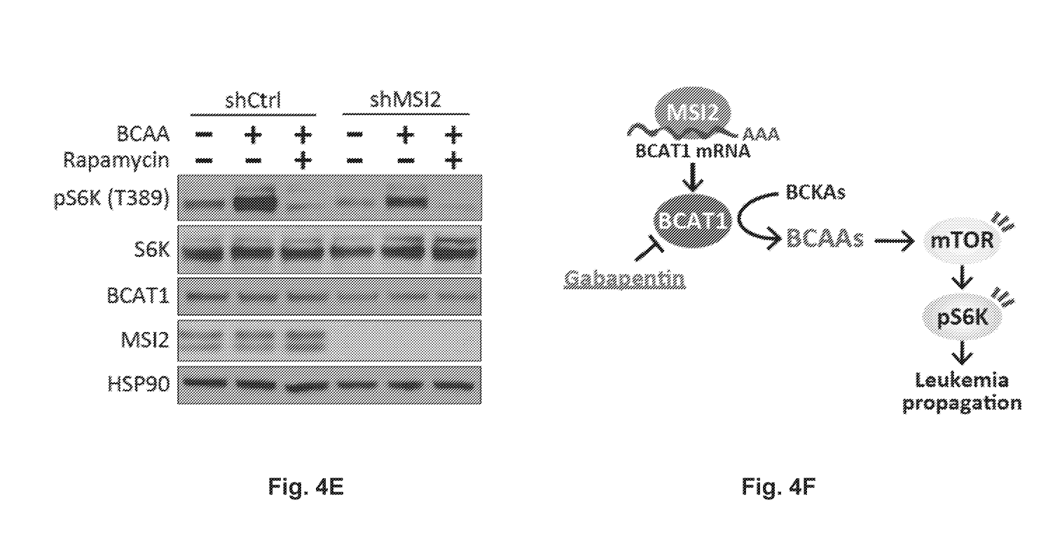

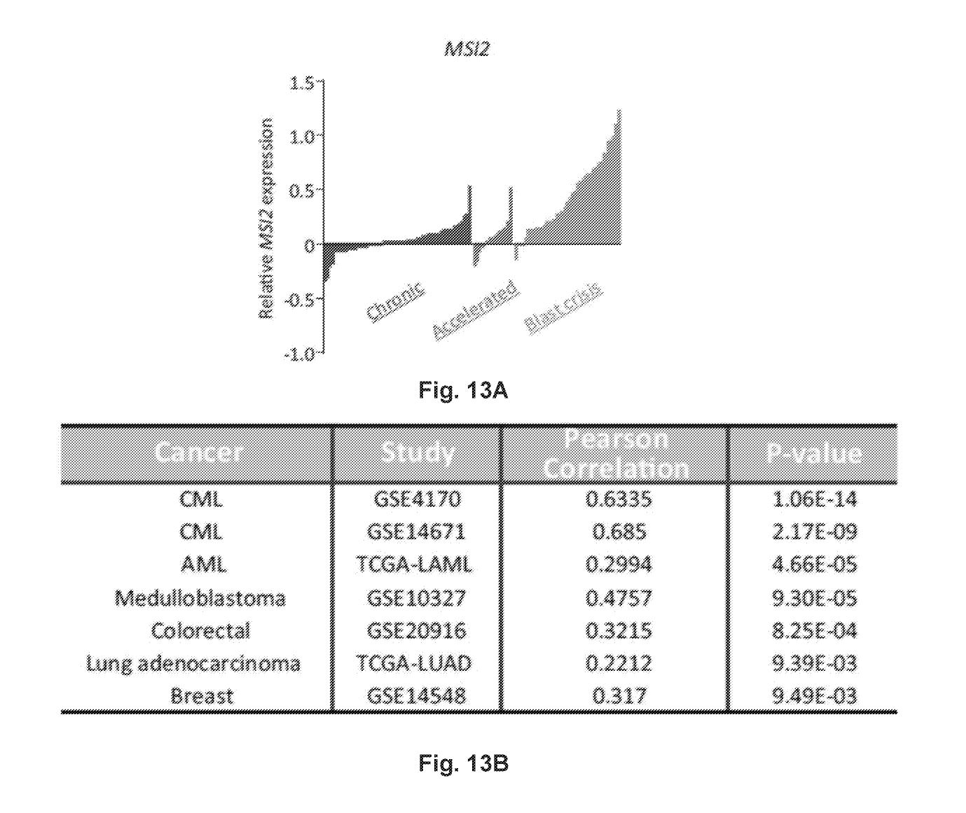

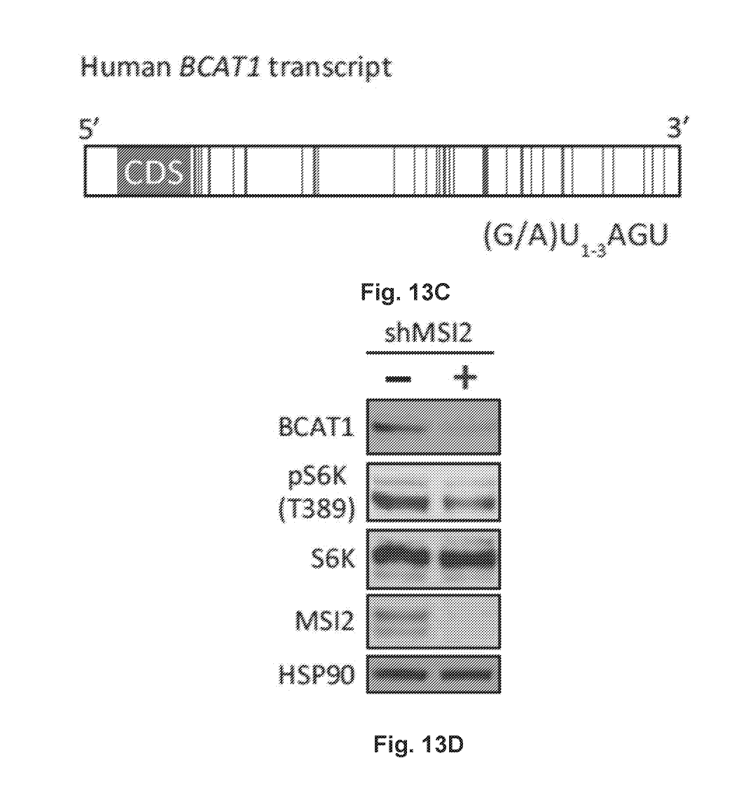

To further investigate the BCAT1-mediated regulation of CML progression, we performed gene correlation analyses using tumor gene expression datasets available in the GEO and TCGA databases. It was found that BCAT1 and MSI2 are often co-expressed in several types of cancer, including leukemias, colorectal and breast cancers (FIGS. 13A-B). MSI2 is a member of the evolutionarily conserved Musashi RNA binding protein family, which regulates cell fates during development and in multiple adult stem cell systems in metazoans¹³⁻¹⁵. At the molecular level, Musashi proteins bind to r(G/A)U₁₋₃AGU sequences (MSI binding elements, MBEs) and post-transcriptionally regulate gene expression via mRNA binding^{16,17}. Importantly, MSI genes are aberrantly activated in human malignancies, such as gliomas and breast and colorectal cancers^{18,19}. In human BC-CML, the MSI2 gene is up-regulated and functionally required for the progression of this leukemia^{20,21}. To determine whether BCAT1 is a direct target of the MSI2 RNA binding protein, the BCAT1 mRNA sequence was analyzed and 40 putative MBEs found in the 3′-untranslated region (3′-UTR; FIG. 13C). To test whether MSI2 binds to the BCAT1 transcripts, a FLAG-tagged MSI2 protein in K562 cells was expressed and RNA immunoprecipitation (RIP) performed. FLAG-MSI2 co-precipitated the BCAT1 transcripts with a >1,500-fold enrichment relative to the vector control (FIG. 4A). In contrast, when RIP was performed with a mutant MSI2 protein in which three phenylalanine residues essential for RNA binding were replaced with leucine¹⁶, the amount of the BCAT1 mRNA recovered was markedly diminished (FIG. 4A, RBD), indicating that the co-precipitation of BCAT1 transcript requires the RNA binding activity of MSI2. The transcripts for beta-2-microglobulin (B2M) or c-Myc oncogene (MYC) contain only one copy of a putative MBE in their 3′-UTRs (data not shown), and MSI2 RIP did not enrich B2M or MYC mRNAs as efficiently as BCAT1 (FIG. 4A). Furthermore, RIP with an anti-MSI2 antibody showed that endogenous MSI2 proteins bound to BCAT1 transcripts, while B2M or MYC mRNAs exhibited minimal enrichment relative to that of an IgG control (FIG. 4B), indicating that MSI2 is specifically associated with the BCAT1 transcripts. Because MSI2 knockdown reduced the levels of BCAT1 protein and phospho-S6K (FIG. 13D), the binding of MSI2 to BCAT1 mRNA positively regulates BCAT1 translation and mTORC1 activation. Importantly, BCAT1 over-expression (FIG. 4C) and BCAA supplementation (FIG. 4D) effectively suppressed the attenuation of the colony-forming ability caused by MSI2 knockdown, with a concomitant increase in pS6K levels in a rapamycin-sensitive manner (FIG. 4E). The levels of AKT phosphorylation were unaffected by shMSI2 (FIG. 12C). Collectively, embodiments of the present disclosure demonstrate an essential role for the MSI2-BCAT1 axis in myeloid leukemia and provides a proof-of-principle for inhibiting the BCAA metabolic pathway to regulate CML progression (FIG. 4F).

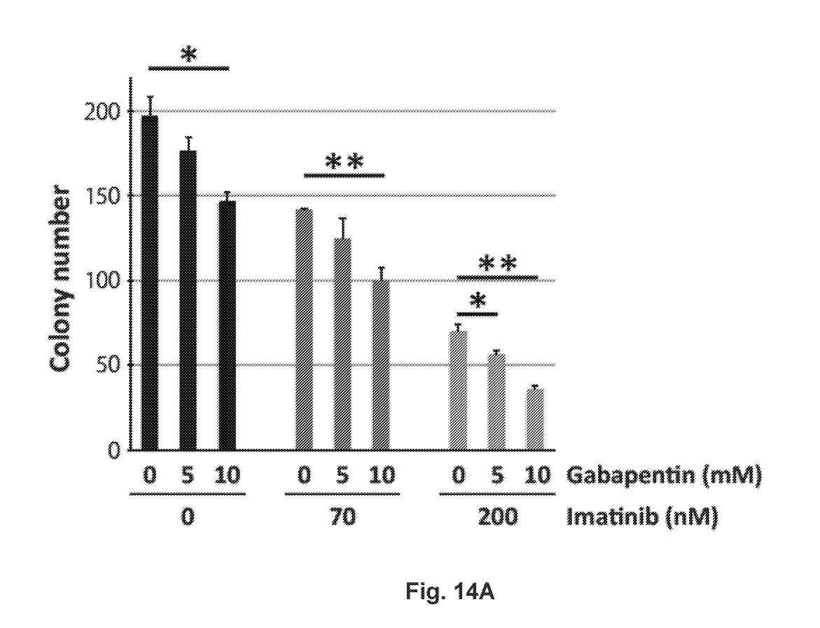

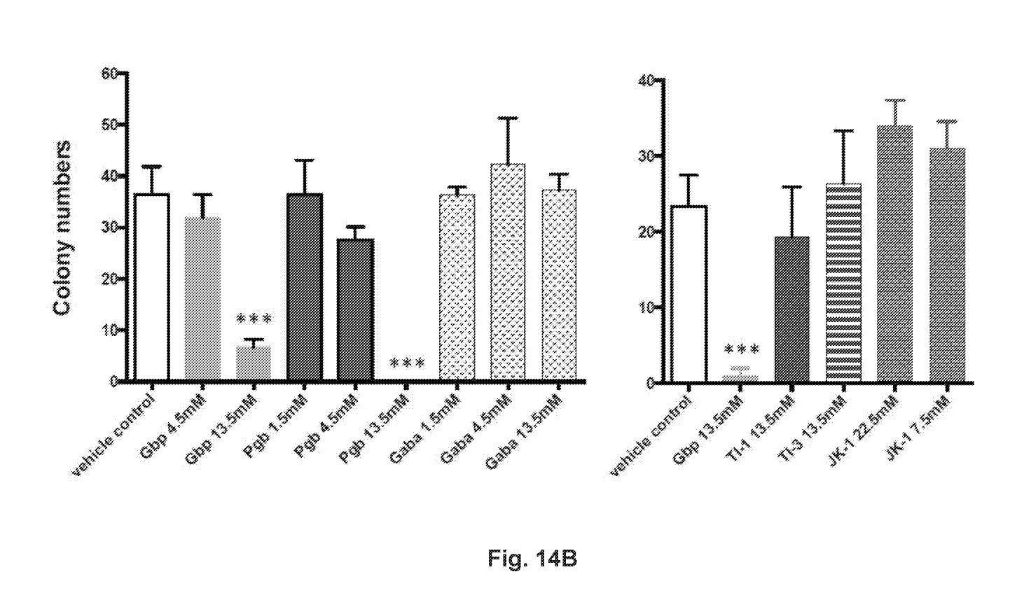

BCAT1 inhibition by Gabapentin is shown in FIG. 14A, leukemic colony formation by human blast crisis CML was attenuated in combination with the tyrosine kinase inhibitor Imatinib. FIG. 14A demonstrates colony-forming ability of K562 human CML cells treated with the indicated concentrations of Gabapentin and Imatinib. Three hundred cells were plated per well in triplicate, and colonies were scored on day 5 (error bars indicates s.e.m. *p<0.05, **p<0.01). Inhibition of leukemic colony formation by gabapentin (Gbp) and its structural analogs was also investigated (FIG. 14B). The colony-forming ability of K562 human CML cells treated with the indicated chemicals was investigated. One hundred fifty cells were plated per well in triplicate, and colonies were scored on day 14 (error bars indicates s.e.m. ***p<0.001 compared with the vehicle control). These results indicate that selective inhibitory activities of Gbp-related amino acid derivatives on the clonogenic growth potentials of K562 leukemia cells.

The up-regulation and functional requirements of BCAT1 have been reported in glioblastoma and in colorectal and breast tumors^{22,23}. Interestingly, Musashi proteins also regulate the same spectrum of cancers including myeloid leukemia^{18-21,24,25}, suggesting a highly conserved role for the MSI-BCAT1 pathway in multiple cancer types. Despite the conservation of this pathway, the metabolic role of BCAT1 seems distinct and dependent on the tissue of origin; in the brain, BCAT1 catalyzes BCAA breakdown and glutamate production to enhance tumor growth in glioblastoma²³, whereas BCAT1 promotes BCAA production in leukemia. Mayers et al. recently showed that two different types of tumors, specifically pancreatic ductal adenocarcinoma (PDAC) and non-small cell lung carcinoma (NSCLC)²⁶, exhibit different usages of BCAAs. Despite the same initiating events of Kras activation and p53 deletion, NSCLC cells actively utilize BCAAs by enhancing their uptake and oxidative breakdown to BCKAs, whereas PDAC cells display decreased uptake and thus little dependency on BCAAs. Consistently, BCAT1 and BCAT2 are required for tumor formation in NSCLC but not in PDAC. Although BCAT1 is functionally required for tumor growth in a broad range of malignancies, these reports and the present disclosure highlight the context-dependent role of the BCAT1 metabolic pathway in cancer.

### Methods

### Mice:

C57BL6/J mice were from the Jackson Laboratory. Mice were bred and maintained in the facility of the University Research Animal Resources at University of Georgia. All mice were 8-16 weeks old, age- and sex-matched and randomly chosen for experimental use. No statistical methods were used for sample size estimates. All animal experiments were performed according to protocols approved by the University of Georgia Institutional Animal Care and Use Committee.

### Cell Isolation, Analysis and Sorting:

Cells were suspended for cell sorting in Hanks' balanced salt solution (HBSS) containing 5% (vol/vol) fetal bovine serum (FBS) and 2 mM EDTA as previously described²⁷. The following antibodies were used to define lineage positive cells: 145-2C11 (CD3ε), GK1.5 (CD4), 53-6.7 (CD8), RB6-8C5 (Ly-6G/Gr1), M1/70 (CD11b/Mac-1), TER119 (Ly-76/TER119), 6B2 (CD45R/B220), and eBio1D3 (CD19). Red blood cells were lysed with RBC Lysis Buffer (eBioscience) before staining for lineage markers. For the Lin⁻ Sca-1⁺ cKit⁺ (LSK) bone marrow cell sorting, the antibodies 2B8 (cKit/CD117) and D7 (Sca-1/Ly-6A/E) antibodies were also used. To determine donor-derived chimerism in the transplantation-based assays, peripheral blood from the recipients was obtained by the submandibular bleeding method and prepared for analysis as previously described²⁰. All antibodies were purchased from eBioscience. Apoptosis assays were performed by staining cells with Annexin V and 7-AAD (BioLegend). Cell cycle status was analyzed by staining cells with 2.5 μg/ml PI containing 0.1% BSA and 2 μg/ml RNase after fixation with 70% ethanol. Flow cytometric analysis and cell sorting were carried out on the Moflo XDP, Cyan ADP (Beckman Coulter) or S3 (Bio-Rad), and the data were analyzed with FlowJo software (Tree Star Inc.).

### Viral Constructs and Production:

Retroviral BCR-ABL1 and NUP98-HOXA9 vectors and lentiviral FG12-UbiC-GFP vector were obtained from Addgene. Mouse Bcat1 cDNA (IMAGE clone ID 30063465) was cloned into MSCV-IRES-GFP and Human BCAT1 cDNA (NITE clone ID AK056255) was cloned into FG12-Ubc-hCD2. The short hairpin RNA constructs against Bcat1 (shBcat1) were designed and cloned in MSCV-LTRmiR30-PIG (LMP) vector from Open Biosystems or TtRMPVIR from Addgene according to their instructions. The target sequences are (SEQ ID 1) 5′-CCCAGTCTCTGATATTCTGTAC-3′ for shBcat1-a, (SEQ ID 2) 5′-TCCGCGCCGTTTGCTGGAGAAA-3′ for shBcat1-b and (SEQ ID 3) 5′-CTGTGCCAGAGTCCTTCGATAG-3′ for luciferase as a negative control (shCtrl). Lentiviral short hairpin RNA (shRNA) constructs were cloned in FG12 essentially as described previously²⁸. The target sequences are (SEQ ID 4) 5′-CGCAGAGTGTACCGGAGA-3′ for shBCAT1-c, (SEQ ID 5) 5′-TGCCCAATGTGAAGCAGT-3′ for shBcat1-d and (SEQ ID 6) 5′-TGCGCTGCTGGTGCCAAC-3′ for luciferase as a negative control. Virus was produced in 293FT cells transfected using polyethylenimine with viral constructs along with VSV-G and gag-pol. For lentivirus production Rev was also co-transfected. Viral supernatants were collected for two days followed by ultracentrifugal concentration at 50,000×g for 2 h.

### Cell Culture and Colony Formation Assays:

The human BC-CML cell line K562, the human acute leukemia cell lines MV4-11 and U937 were maintained in Roswell Park Memorial Institute 1640 medium (RPMI-1640) with 10% FBS, 100 IU/ml penicillin and 100 μg/ml streptomycin. The human acute promyelocytic leukemia cell line HL60 was maintained in RPMI supplemented with 20% FBS. All human cell lines were obtained from ATCC, and cell line authentication testing was performed by ATCC-standardized STR analysis to verify their identity in July 2016. For the colony forming assays, the cells were transduced with lentiviral shRNA and plated in triplicate in 1.2% methylcellulose medium (R&D systems) supplemented with 100 IU/ml penicillin and 100 μg/ml streptomycin, 10% FBS. Where indicated, either BCAAs (L-Leucine, L-Valine, L-Isoleucine, 4 mM each, Sigma-Aldrich), L-alanyl-L-glutamine (4 mM, GlutaMax™, Life Technologies), rapamycin (50 nM, Tocris) or gabapentin (Gbp; Tokyo Chemical Industry Co.) was added to the medium. Gbp was freshly dissolved in PBS before use. Colonies were scored on days 9 to 14. For liquid culture of murine cells, freshly isolated adult LSK cells or Lin⁻ BC-CML cells were plated into a 96-well U bottom plate in X-Vivo15 (with Gentamicin and Phenol Red; Lonza) supplemented with 50 μM 2-mercaptoethanol, 10% FBS, 100 ng/ml stem cell factor (SCF, eBioscience) and 20 ng/ml thrombopoietin (TPO, Peprotech). For the BC-CML and LSK colony formation assays, BCR-ABL⁺ NUP98-HOXA9⁺ or infected construct-positive cells were sorted and plated in triplicate in Iscove's modified medium (IMDM)-based methylcellulose medium (Methocult M3434, StemCell Technologies). Colonies were scored on days 7 to 10.

### Generation and Analysis of Leukemic Mice:

Mice bearing CP- and BC-CML were generated essentially as previously described^{3,4,29-31}. In brief, CP-CML was modeled by transducing the oncogene BCR-ABL1 into hematopoietic stem/progenitor cells (HSPCs) defined by the LSK surface marker phenotype from normal bone marrow, which were transplanted into conditioned recipient mice. BC-CML was modeled by transplanting LSK cells infected with two oncogenes, BCR-ABL1 and NUP98-HOXA9, which are associated with myeloid BC-CML in humans. LSK cells were sorted from healthy C57BL6/J bone marrow and cultured in X-Vivo15 media supplemented with 50 μM 2-mercaptoethanol, 10% FBS, 100 ng/ml SCF and 20 ng/ml TPO. After incubation overnight, cells were infected with retroviruses carrying the oncogenes. Viruses used were as follows: MSCV-BCR-ABL-IRES-YFP to generate CP-CML, or MSCV-BCR-ABL-IRES-YFP and MSCV-NUP98-HOXA9-IRES-tNGFR to generate BC-CML. Cells were harvested 48 h after infection and transplanted retro-orbitally into groups of C57BL6/J mice. Recipients were lethally irradiated (9.5 Gy) for CP-CML and sublethally (6 Gy) for BC-CML. For Bcat1 overexpression, LSK cells were infected with MSCV-BCR-ABL-IRES-YFP and MSCV-Bcat1-IRES-GFP, and doubly infected cells were FACS-purified and transplanted into recipients that were sublethally irradiated. For Bcat1 knockdown by retroviral shRNA transduction, the Lin⁻ population from BC-CML cells was sorted and infected with either control shCtrl (against luciferase) or shBcat1-a/b (against Bcat1) retrovirus for 48 h. Infected cells were sorted based on GFP expression, and 1,000 or 2,000 cells were transplanted in sublethally irradiated C57BL6/J recipients. For conditional Bcat1 knockdown by a Dox-inducible shRNA system, animals were analyzed for donor chimerism at day 10 post-transplantation, and then Dox treatment was initiated by feeding Dox-containing rodent chow (0.2 mg/g diet; S3888, BioServ). After transplantation, recipient mice were maintained on antibiotic water (sulfamethoxazole/trimethoprim) and evaluated daily for signs of morbidity, weight loss, failure to groom, and splenomegaly. Premorbid animals were sacrificed, and relevant tissues were harvested and analyzed by flow cytometry and histopathology. For secondary BC-CML transplantations, cells recovered from terminally ill primary recipients were sorted for Lin⁻ donor cells and transplanted into secondary recipients. Where indicated, sorted live BC-CML cells from the spleen were cytospun and stained with Wright's stain solution (Harleco) for cytopathologic evaluation by a board-certified veterinary pathologist.

### Primary Human Leukemia Samples:

Patient blood samples were obtained at the Institute of Medical Science, the University of Tokyo (IMSUT) Hospital with written informed consent according to the procedures approved by the Institutional Review Board. Mononuclear cells from the subjects were viably frozen and stored in liquid nitrogen. For in vitro colony formation with BCAT1 knockdown, primary hCD34⁺ cells sorted from patient bone marrow samples were cultured in IMDM supplemented with 10% FBS, 100 IU/ml penicillin and 100 μg/ml streptomycin, 55 μM 2-mercaptoethanol, SCF, IL-3, IL-6, FLT3L and TPO. After 24 h of culture, the cells were transduced with lentiviral shRNA (cloned in FG12-UbiC-GFP), and the GFP-positive infected cells were sorted at 48 h, and 5,000-50,000 cells were plated in complete methylcellulose medium (Methocult H4435, StemCell Technologies). For the colony forming assays with Gbp, sorted hCD34⁺ cells from the primary patient specimens were cultured in complete methylcellulose medium with the indicated concentrations of Gbp. Colonies were scored on days 9 to 14.

### Bioinformatic Analysis of Human Gene Expression:

For the focused gene expression analysis of BCAT1, BCAT2 and MSI2 in human CML progression, the GEO dataset GSE4170 was retrieved and analyzed using Python v2.7 and the SciPy statistical toolkit. Pearson correlation coefficients were used to find patterns of co-expression. For co-expression analysis of BCAT1 and MSI2 across multiple cancer types, the GEO datasets GSE14671 (CML), GSE10327 (medulloblastoma), GSE20916 (colorectal), GSE14548 (breast) and TCGA datasets LAML (AML) and LUAD (lung adenocarcinoma) were collected and analyzed in a similar fashion.

### Realtime and Standard RT-PCR Analysis:

Total cellular RNAs were isolated using RNAqueous-Micro kit (Ambion) and cDNAs were prepared from equal amounts of RNAs using Superscript III reverse transcriptase (Life Technologies). For standard PCRs, the reactions were performed with DreamTaq PCR Master Mix (Life Technologies), cDNA and 0.5 μM of each primer. PCR conditions were as follows: 1 min at 94° C., followed by 35 cycles at 94° C. for 30 s, 58° C. for 30 s, and 72° C. for 30 s. PCR primer sequences are as follows:

### (SEQ ID 7) B2m-F1, 5′-ACCGGCCTGTATGCTATCCAGAA-3′; (SEQ ID 8) B2m-R2, 5′-CCATACTGGCATGCTTAACTCTG-3′;

(SEQ ID 9) Bcat1-F1, 5′-TGTGGCTGTACGGCAAGGACAAC-3′; (SEQ ID 10) Bcat1-R2, 5′-GTAGCTCGATTGTCCAGTCACT-3′. Quantitative real-time PCRs were performed using EvaGreen® qPCR Master Mix (Bio-Rad) on an iQ5 (Bio-Rad), or using TaqMan Gene Expression Assays on an Applied Biosystems® 7500 Real-Time PCR Systems (Life Technologies). Results were normalized to the level of β-2-microglobulin. PCR primer sequences are as follows:
(SEQ ID 11) mB2m-F, 5′-ACCGGCCTGTATGCTATCCAGAA-3′; (SEQ ID 12) mB2m-R, 5′-AATGTGAGGCGGGTGGAACTGT-3′;
(SEQ ID 13) hB2M-F, 5′-ATGAGTATGCCTGCCGTGTGA-3′; (SEQ ID 14) hB2M-R, 5′-GGCATCTTCAAACCTCCATG-3′;
(SEQ ID 15) hBCAT1-F, 5′-TGGAGAATGGTCCTAAGCTG-3′; (SEQ ID 16) hBCAT1-R, 5′-GCACAATTGTCCAGTCGCTC-3′;
(SEQ ID 17) hMYC-F, 5′-GAGCAAGGACGCGACTCTCC-3′; (SEQ ID 18) hMYC-R, 5′-GCACCGAGTCGTAGTCGAGG-3′. The following genes were analyzed with TaqMan Gene Expression Assays: Bcat1 (Mm00500289_m1), Bcat2 (Mm00802192_m1), Gpt1 (Mm00805379_g1), Gpt2 (Mm00558028_m1), Got1 (Mm00494698_m1), Got2 (Mm00494703_m1).

### Amino Acid and Keto Acid Quantification:

Leukemia cells or peripheral blood samples drawn from mice bearing myeloid leukemia were used for amino acid and keto acid analysis by high-performance liquid chromatography (HPLC)-fluorescence detection, as described³²⁻³⁴. In brief, two hundred thousand live leukemia cells per sample were sorted and washed twice with ice-cold PBS to remove media components prior to amino acid extraction. The blood plasma was prepared by centrifugation of the peripheral blood samples at 2,000×g at 4° C. for 10 min. Plasma fractions were then treated with 45% methanol/45% acetonitrile containing 6-aminocaproic acid (internal standard for amino acid analysis) or α-ketovalerate (internal standard for keto acid analysis) on ice for 10 min. Cell samples were treated with 80% methanol instead of 45% methanol/acetonitrile mixture. After removing the insoluble particles by centrifugation, the supernatants were collected and dried using a SpeedVac at 30-45° C. For amino acid quantification, the dried samples were treated with the amine-reactive 4-fluoro-7-nitro-2,1,3-benzoxadizole (NBD-F) to derivatize the amino acids. HPLC separation of NBD-amino acids was carried out on an Inertsil ODS-4 column (3.0×250 mm, 5 μm, GL Sciences, Tokyo, Japan) at a flow rate of 0.6 ml min⁻¹. We used two types of mobile phase conditions for the separation of 16 amino acids. The mobile phases included (A) 25 mM citrate buffer containing 25 mM sodium perchlorate (pH 6.2) and (B) water/acetonitrile (50/50, v/v). The gradient conditions were as follows: t=0 min, 10% B; t=20 min, 50% B; and t=30 min, 100% B. For NBD-Asn, Ser, Thr, Gln and Phe analysis, 25 mM citrate buffer containing 25 mM sodium perchlorate (pH 4.4) was used as the mobile phase A. NBD-amino acids were detected with excitation and emission wavelengths of 470 and 530 nm, respectively. For keto acid quantification, dried samples were treated with o-phenylenediamine (OPD) to derivatize alpha-keto acids, followed by liquid-liquid extraction with ethyl acetate. HPLC separation of OPD-keto acids was carried out on an Inertsil ODS-4 column (3.0×250 mm, 5 μm) at a flow rate of 0.6 ml min⁻¹. Mobile phase was water/methanol (55/45, v/v). The fluorescence detection was carried out at the emission wavelength of 410 nm with excitation of 350 nm.

### Measurement of Leucine Uptake in Primary Mouse Leukemia Cells:

Primary mouse leukemia cells from the spleens of the mice bearing myeloid leukemia were used for the analysis of leucine uptake essentially as described previously^{35,36}. In brief, live leukemia cells were sorted and washed with HBSS to remove media components. The cells were incubated at 37° C. for 1-3 min with pre-warmed HBSS containing 10 μM [(U)-¹⁴C]-L-leucine (Moravek Inc., specific activity, 328 mCi/mmol). The cells were subsequently washed twice with cold HBSS and lysed using 100 mM NaOH. The solubilized cell lysates were mixed with the EcoLume liquid scintillation cocktail (MP Biomedicals), and radioactivity was measured using an L56500 liquid scintillation counter (Beckman Coulter). Leucine uptake was quantified using a calibration curve of [¹⁴C]-L-leucine reference standard samples.

### NMR-Based Metabolic Analysis:

Cells were cultured and labeled in media supplemented with either 170 μM [(U)-¹³C]-L-valine, 30 or 170 μM [(U)-¹³C]-ketoisovalerate (KIV) sodium salt (for ¹³C tracer experiments; Cambridge Isotope Laboratories) or 2 mM [amine-¹⁵N]-L-glutamine (for ¹⁵N tracer experiments; Cambridge Isotope Laboratories). The concentrations are based on the standard RPMI-1640 media formulation. At the time of collection, the cells were washed twice with ice-cold PBS and extracted with 80% methanol on ice for 10 min. After removing the insoluble particles by centrifugation, the supernatants were collected and dried using a SpeedVac at 30° C. The cell extracts were dissolved in a total volume of 90 μL 99.96% D₂O containing 0.1 mM DSS-d6 and transferred to 3-mm NMR tubes (Shigemi Inc.). Calibration samples (150-250 mM) were prepared from 98% ¹⁵N-enriched glutamine, glutamic acid, valine, leucine, isoleucine and alanine (Isotec Inc.) and ¹³C-enriched KIV and ¹³C,¹⁵N-enriched valine (Cambridge Isotope Laboratories) in D₂O containing 0.1 mM DSS. All signals were identified either with authentic samples or by reference to literature values. Two-dimensional proton correlated spectra (COSY and TOCSY) were also collected in some cases to confirm assignments. The data were collected at 25° C. on Agilent DD2 spectrometers at 600 or 900 MHz equipped with cryogenically cooled probes. The ¹H data were collected with a 20-sec relaxation delay for accurate integration. The ¹⁵N data were acquired with a two-dimensional heteronuclear multiple bond correlation experiment (gNhmbc) derived from the Agilent pulse program library with the transfer delay set for a ¹⁵N-¹H coupling value of 4 Hz. Typically, data sets were 2000×64 complex points with the ¹⁵N dimension set between 30 and 46 ppm, and 64 scans per point. The ¹³C data were acquired with a two-dimensional heteronuclear single bond correlation experiment (HSQCAD) from the Agilent pulse program library, and the datasets were 1202×64 complex points with the ¹³C dimension set between 10 and 80 ppm with 16 scans per point. One-dimensional spectra were also collected using the same heteronuclear correlation experiments for ¹⁵N and ¹³C. The data were processed using MestReNova software (Mestrelab Research S.L.). One-dimensional proton data were processed with 0.3 Hz line broadening and polynomial baseline correction. The gNhmbc and HSQC data were processed with linear prediction and zero-filling in the ¹⁵N and ¹³C dimensions. Integration was achieved by summing over peak areas with the contribution of noise subtracted in the ¹⁵N spectra. To calculate the concentrations in the ¹⁵N tracer experiments, the ¹H and gNhmbc spectra of the calibration samples were integrated, and a scaling factor was derived from the ratio of the known concentration of each 98% enriched ¹⁵N-amino acid and the integral values from the gNhmbc data. These factors are a function of the 3-bond coupling between the ¹⁵N-amine and β-protons as well as the number of those protons. Therefore, the concentrations of each amino acid in cell extracts can be estimated from their integral values by applying the respective scaling factor. For quantification of ¹³C-labeled compounds, the methyl groups in the ¹H and HSQC spectra of the calibration references were integrated, and a scaling factor was derived essentially as described above and used to calculate concentrations from the HSQC data of each sample.

### Antibodies:

Anti-FLAG monoclonal antibody M2 (Sigma-Aldrich), anti-MSI2 monoclonal antibody EP1305Y (Abcam) and normal Rabbit IgG PP64B (Millipore) were used for immunoprecipitation. For Western blotting the following antibodies were used: mouse monoclonal BCAT1 (clone ECA39, BD Transduction Laboratories) and Bcat1 OTI3F5 (OriGene), rabbit monoclonal S6K (#9202 and #2708), pS6K (#9234), AKT (#4691), pAKT, T308 (#13038) and pAKT, S473 (#4060) from Cell Signaling, rabbit monoclonal MSI2 EP1305Y, mouse monoclonal HSP90 F-8 (Santa Cruz Biotech) and mouse monoclonal β-tubulin BT7R (Thermo Fisher Scientific).

### RNA Immunoprecipitation Assays:

K562 cells were lysed in 50 mM Tris/HCl (pH 7.5) containing 150 mM NaCl, 5 mM EDTA, 1% NP-40, and the Halt Protease and Phosphatase Inhibitor Cocktail (Thermo Fisher Scientific). We performed immunoprecipitations with anti-FLAG, anti-MSI2 or rabbit normal IgG and protein G magnetic beads (Life Technologies) for 1 h at 4° C. The immunoprecipitated protein-RNA complexes were washed three times with low- and high-salt wash buffers (300 mM or 550 mM NaCl, respectively), followed by three washes in PBS. Total RNAs were purified from the washed beads using the RNAqueous-Micro kit (Ambion) and subjected to RT-qPCR analysis for quantification. The fold enrichment of the transcript amount in the RIP fraction over the amount present in the input sample before RIP (RIP/input) was calculated for each sample.

### Statistical Analysis:

Statistical analyses were carried out using GraphPad Prism software version 6.0f (GraphPad Software Inc.). Data are shown as the mean±the s.e.m. Two-tailed unpaired Student's t-tests or Mann-Whitney U tests were used to determine statistical significance. For Kaplan Meier survival analysis, log-rank tests were used for statistical significance (*p<0.05, **p<0.01, ***p<0.001).

### Summary of Sequences

| ID number | Sequence | Name | Source Organism |
|---|---|---|---|
| (SEQ ID 1) | 5′-CCCAGTCTCTGATATTCTGTAC-3′ | shBcat1-a | Mus musculus |
| (SEQ ID 2) | 5′-TCCGCGCCGTTTGCTGGAGAAA- | shBcat1-b | Mus musculus |
| | 3′ | | |
| (SEQ ID 3) | 5′-CTGTGCCAGAGTCCTTCGATAG-3′ | luciferase as | Artificial from |
| | | a negative | Lampyridae |
| | | control (shCtrl) | |
| (SEQ ID 4) | 5′-CGCAGAGTGTACCGGAGA-3′ | shBCAT1-c | Human |
| (SEQ ID 5) | 5′-TGCCCAATGTGAAGCAGT-3′ | shBCAT1-d | Human |
| (SEQ ID 6) | 5′-TGCGCTGCTGGTGCCAAC-3′ | | Artificial from |
| | | | Lampyridae |
| (SEQ ID 7) | 5′-ACCGGCCTGTATGCTATCCAGAA- | B2m-F1 | Artificial |
| | 3′ | | Sequence PCR |
| | | | Forward Primer |
| (SEQ ID 8) | 5′-CCATACTGGCATGCTTAACTCTG- | B2m-R2 | Artificial |
| | 3′ | | Sequence PCR |
| | | | Reverse Primer |
| (SEQ ID 9) | 5′- | Bcat1-F1 | Artificial |
| | TGTGGCTGTACGGCAAGGACAAC-3′ | | Sequence PCR |
| | | | Forward Primer |
| (SEQ ID 10) | 5′-GTAGCTCGATTGTCCAGTCACT-3′ | Bcat1-R2 | Artificial |
| | | | Sequence PCR |
| | | | Reverse Primer |
| (SEQ ID 11) | 5′- | mB2m-F | Artificial |
| | ACCGGCCTGTATGCTATCCAGAA-3′ | | Sequence PCR |
| | | | Forward Primer |
| (SEQ ID 12) | 5′-AATGTGAGGCGGGTGGAACTGT- | mB2m-R | Artificial |
| | 3′ | | Sequence PCR |
| | | | Reverse Primer |
| (SEQ ID 13) | 5′-ATGAGTATGCCTGCCGTGTGA-3′ | hB2M-F | Artificial |
| | | | Sequence PCR |
| | | | Forward Primer |
| (SEQ ID 14) | 5′-GGCATCTTCAAACCTCCATG-3′ | hB2M-R | Artificial |
| | | | Sequence PCR |
| | | | Reverse Primer |
| (SEQ ID 15) | 5′-TGGAGAATGGTCCTAAGCTG-3′ | hBCAT1-F | Artificial |
| | | | Sequence PCR |
| | | | Forward Primer |
| (SEQ ID 16) | 5′-GCACAATTGTCCAGTCGCTC-3′ | hBCAT1-R | Artificial |
| | | | Sequence PCR |
| | | | Reverse Primer |
| (SEQ ID 17) | 5′-GAGCAAGGACGCGACTCTCC-3′ | hMYC-F | Artificial |
| | | | Sequence PCR |
| | | | Forward Primer |
| (SEQ ID 18) | 5′-GCACCGAGTCGTAGTCGAGG-3′ | hMYC-R | Artificial |
| | | | Sequence PCR |
| | | | Reverse Primer |

### REFERENCES

1. Vander Heiden, M. G. Targeting cancer metabolism: a therapeutic window opens. Nat Rev Drug Discov 10, 671-684 (2011).
2. DeBerardinis, R. J. & Thompson, C. B. Cellular metabolism and disease: what do metabolic outliers teach us? Cell 148, 1132-1144 (2012).
3. Daley, G., Van Etten, R. & Baltimore, D. Induction of chronic myelogenous leukemia in mice by the P210bcr/abl gene of the Philadelphia chromosome. Science 247, 824-830 (1990).
4. Dash, A. B. et al. A murine model of CML blast crisis induced by cooperation between BCR/ABL and NUP98/HOXA9. Proc Natl Acad Sci USA 99, 7622-7627 (2002).
5. Brosnan, J. T. & Brosnan, M. E. Branched-chain amino acids: enzyme and substrate regulation. J. Nutr. 136, 207S-11S (2006).
6. Hutson, S. M., Sweatt, A. J. & Lanoue, K. F. Branched-chain amino acid metabolism: implications for establishing safe intakes. J. Nutr. 135, 1557S-64S (2005).
7. Hutson, S. M. et al. Role of branched-chain aminotransferase isoenzymes and gabapentin in neurotransmitter metabolism. J Neurochem 71, 863-874 (1998).
8. Radich, J. P. et al. Gene expression changes associated with progression and response in chronic myeloid leukemia. Proc Natl Acad Sci USA 103, 2794-2799 (2006).
9. Wolfson, R. L. et al. Sestrin2 is a leucine sensor for the mTORC1 pathway. Science 351, 43-48 (2016).
10. Han, J. M. et al. Leucyl-tRNA synthetase is an intracellular leucine sensor for the mTORC1-signaling pathway. Cell 149, 410-424 (2012).
11. Bar-Peled, L. & Sabatini, D. M. Regulation of mTORC1 by amino acids. Trends Cell Biol. 24, 400-406 (2014).
12. Jewell, J. L., Russell, R. C. & Guan, K.-L. Amino acid signalling upstream of mTOR. Nat. Rev. Mol. Cell Biol. 14, 133-139 (2013).
13. Okano, H., Imai, T. & Okabe, M. Musashi: a translational regulator of cell fate. J. Cell. Sci. 115, 1355-1359 (2002).
14. Sutherland, J. M., McLaughlin, E. A., Hime, G. R. & Siddall, N. A. The Musashi family of RNA binding proteins: master regulators of multiple stem cell populations. Adv. Exp. Med. Biol. 786, 233-245 (2013).
15. Rentas, S. et al. Musashi-2 attenuates AHR signalling to expand human haematopoietic stem cells. 532, 508-511 (2016).
16. Imai, T. et al. The neural RNA-binding protein Musashi1 translationally regulates mammalian numb gene expression by interacting with its mRNA. Mol Cell Biol 21, 3888-3900 (2001).
17. Cragle, C. & MacNicol, A. M. Musashi protein-directed translational activation of target mRNAs is mediated by the poly(A) polymerase, germ line development defective-2. J Biol Chem 289, 14239-14251 (2014).
18. Wang, X.-Y. et al. Musashi1 regulates breast tumor cell proliferation and is a prognostic indicator of poor survival. Mol Cancer 9, 221 (2010).
19. Muto, J. et al. RNA-Binding Protein Musashi1 Modulates Glioma Cell Growth through the Post-Transcriptional Regulation of Notch and PI3 Kinase/Akt Signaling Pathways. PLoS ONE 7, e33431 (2012).
20. Ito, T. et al. Regulation of myeloid leukaemia by the cell-fate determinant Musashi. Nature 466, 765-768 (2010).
21. Kharas, M. G. et al. Musashi-2 regulates normal hematopoiesis and promotes aggressive myeloid leukemia. Nat Med 16, 903-908 (2010).
22. Yoshikawa, R. et al. ECA39 is a novel distant metastasis-related biomarker in colorectal cancer. World J Gastroenterol 12, 5884-5889 (2006).
23. Tönjes, M. et al. BCAT1 promotes cell proliferation through amino acid catabolism in gliomas carrying wild-type IDH1. Nat Med 19, 901-908 (2013).
24. Lagadec, C. et al. The RNA-binding protein Musashi-1 regulates proteasome subunit expression in breast cancer- and glioma-initiating cells. Stem Cells 32, 135-144 (2014).
25. Li, N. et al. The Msi Family of RNA-Binding Proteins Function Redundantly as Intestinal Oncoproteins. Cell Rep 13, 2440-2455 (2015).
26. Mayers, J. R. et al. Tissue of origin dictates branched-chain amino acid metabolism in mutant Kras-driven cancers. Science 353, 1161-1165 (2016).
27. Domen, J., Cheshier, S. H. & Weissman, I. L. The role of apoptosis in the regulation of hematopoietic stem cells: Overexpression of Bcl-2 increases both their number and repopulation potential. J Exp Med 191, 253-264 (2000).
28. Qin, X.-F., An, D. S., Chen, I. S. & Baltimore, D. Inhibiting HIV-1 infection in human T cells by lentiviral-mediated delivery of small interfering RNA against CCR5. Proc Natl Acad Sci USA 100, 183-188 (2003).
29. Mayotte, N., Roy, D.-C., Yao, J., Kroon, E. & Sauvageau, G. Oncogenic interaction between BCR-ABL and NUP98-HOXA9 demonstrated by the use of an in vitro purging culture system. Blood 100, 4177-4184 (2002).
30. Neering, S. J. et al. Leukemia stem cells in a genetically defined murine model of blast-crisis CML. Blood 110, 2578-2585 (2007).
31. Ashton, J. M. et al. Gene Sets Identified with Oncogene Cooperativity Analysis Regulate In Vivo Growth and Survival of Leukemia Stem Cells. Cell Stem Cell 11, 359-372 (2012).
32. Song, Y., Funatsu, T. & Tsunoda, M. Amino acids analysis using a monolithic silica column after derivatization with 4-fluoro-7-nitro-2,1,3-benzoxadiazole (NBD-F). J. Chromatogr. B Analyt. Technol. Biomed. Life Sci. 879, 335-340 (2011).
33. Song, Y., Funatsu, T. & Tsunoda, M. Rapid determination of amino acids in biological samples using a monolithic silica column. Amino Acids 42, 1897-1902 (2012).
34. Pailla, K., Blonde-Cynober, F., Aussel, C., De Bandt, J. P. & Cynober, L. Branched-chain keto-acids and pyruvate in blood: measurement by HPLC with fluorimetric detection and changes in older subjects. Clin. Chem. 46, 848-853 (2000).
35. Segawa, H. et al. Identification and functional characterization of a Na+-independent neutral amino acid transporter with broad substrate selectivity. J. Biol. Chem. 274, 19745-19751 (1999).
36. Morimoto, E. et al. Establishment and Characterization of Mammalian Cell Lines Stably Expressing Human L-Type Amino Acid Transporters. J Pharmacol Sci 108, 505-516 (2008).
37. Sud, M. et al. Metabolomics Workbench: An international repository for metabolomics data and metadata, metabolite standards, protocols, tutorials and training, and analysis tools. Nucleic Acids Res 44, D463-70 (2016).

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. (canceled)

2. A method of modulating cancer progression and development, comprising administering to a subject a therapeutically effective amount of one or more compositions including a tyrosine kinase inhibitor, gabapentin or derivatives thereof, and rapamycin or derivatives thereof, or a pharmaceutically acceptable salt or a prodrug of one or more of the tyrosine kinase inhibitor, gabapentin or derivatives thereof, and rapamycin or derivatives thereof, wherein the one or more compositions interrupts a branched-chain amino acid transamination pathway.

3. The method of claim 2, wherein the cancer is chronic myeloid leukemia or acute myeloid leukemia.

4. A method of treating myeloid leukemia in a subject, comprising: administering to a subject a therapeutically effective amount of each of a tyrosine kinase inhibitor, gabapentin or derivatives thereof, and rapamycin or derivatives thereof, or a pharmaceutically acceptable salt or a prodrug of one or more of the tyrosine kinase inhibitor, gabapentin or derivatives thereof, and rapamycin or derivatives thereof.

5. The method of claim 4, wherein each of the tyrosine kinase inhibitor, gabapentin or derivatives thereof, and rapamycin or derivatives thereof are administered independently of one another.

6. The method of claim 4, wherein the administration of each of the tyrosine kinase inhibitor, gabapentin or derivatives thereof, and rapamycin or derivatives thereof is conducted simultaneously.

7. (canceled)

8. The method of claim 4, wherein the myeloid leukemia is chronic myeloid leukemia or acute myeloid leukemia.

9. The method of claim 2, wherein the tyrosine kinase inhibitor is selected from the group consisting of: small molecule inhibitors of tyrosine kinases, antibodies to tyrosine kinases, and antisense oligomers and RNAi inhibitors that reduce the expression of tyrosine kinases.

10. A pharmaceutical composition comprising a therapeutically effective amount of a composition including a tyrosine kinase inhibitor, gabapentin or derivatives thereof, and rapamycin or derivatives thereof, or a pharmaceutically acceptable salt or a prodrug of one or more of the tyrosine kinase inhibitor, gabapentin or derivatives thereof, and rapamycin or derivatives thereof, and a pharmaceutically acceptable carrier.

11. The pharmaceutical composition of claim 10, wherein the tyrosine kinase inhibitor is selected from the group consisting of: small molecule inhibitors of tyrosine kinases, antibodies to tyrosine kinases, and antisense oligomers and RNAi inhibitors that reduce the expression of tyrosine kinases.

12. The method of claim 4, wherein the tyrosine kinase inhibitor is selected from the group consisting of: small molecule inhibitors of tyrosine kinases, antibodies to tyrosine kinases, and antisense oligomers and RNAi inhibitors that reduce the expression of tyrosine kinases.

13. The method of claim 3, wherein the chronic myeloid leukemia is in blast crisis condition.

14. The method of claim 2, wherein each of the tyrosine kinase inhibitor, gabapentin or derivatives thereof, and rapamycin or derivatives thereof are administered independently of one another.

15. The method of claim 2, wherein the administration of each of the tyrosine kinase inhibitor, gabapentin or derivatives thereof, and rapamycin or derivatives thereof is conducted simultaneously.

16. The method of claim 4, wherein the cancer is chronic myeloid leukemia or acute myeloid leukemia.

17. The method of claim 8, wherein the chronic myeloid leukemia is in blast crisis condition.
